(19) United States
(12) Patent Application Publication
(10) Pub. No.: US 2025/0161312 A1
(43) Pub. Date: May 22, 2025

(54) A METHOD FOR THE TREATMENT OF CHEMOTHERAPEUTIC DRUG-INDUCED NEPHROTOXICITY

(71) Applicants: INSTITUT NATIONAL DE LA SANTÉ ET DE LA RECHERCHE MÉDICALE, Paris (FR); CENTRE HOSPITALIER UNIVERSITAIRE DE LILLE, Lille (FR); UNIVERSITÉ DE LILLE, Lille (FR)

(72) Inventors: David BLUM, Lille Cedex (FR); Christelle CAUFFIEZ, Lille Cedex (FR); Nicolas POTTIER, Lille Cedex (FR); Edmone DEWAELES, Lille Cedex (FR)

(21) Appl. No.: 18/839,126

(22) PCT Filed: Mar. 7, 2023
(86) PCT No.: PCT/EP2023/055801

§ 371 Date: Aug. 16, 2024

(30) Foreign Application Priority Data
Mar. 8, 2022 (EP) 22305261.4

(51) Int. Cl. A61K 31/522; A61P 35/00

(52) U.S. Cl. CPC A61K 31/522; A61P 35/00

(57) ABSTRACT

Cisplatin is a potent chemotherapeutic drug, widely used in the treatment of various solid cancers. However, its clinical effectiveness is strongly limited by frequent severe adverse effects, such as nephrotoxicity. Therefore, there is an urgent medical need to identify novel strategies limiting cisplatin-induced toxicity. Here, the inventors provide evidence that the FDA-approved adenosine A_{2A} receptor antagonist istradefylline (KW-6002) significantly protects from cisplatin-induced nephrotoxicity in experimental models of acute and sub-chronic cisplatin intoxication. In particular, the present invention relates to a method for the treatment of cisplatin-induced nephrotoxicity in a subject in need therefore comprising administering to the subject a therapeutically effective amount of a selective A_{2A} Adenosine Receptor (A_{2A}R) antagonist.

### FIELD OF THE INVENTION

The invention relates to a method for the treatment of chemotherapeutic drug-induced nephrotoxicity in a subject in need therefore comprising administering to the subject a therapeutically effective amount of a selective A_{2A} Adenosine Receptor (A2AR) antagonist.

### BACKGROUND OF THE INVENTION

Cisplatin is a potent antineoplastic agent widely used in the treatment of various solid cancers such as lung, ovarian or testicular cancers as well as certain forms of lymphomas (1). The anti-tumor action of cisplatin requires its intracellular bioactivation by the replacement of chlorides by water molecules, forming a highly reactive molecule that binds to DNA and induces cytotoxic lesions in tumors (2). However, the unwanted accumulation of cisplatin in healthy cells can also trigger genotoxicity. Indeed, the clinical use of cisplatin is restricted by various severe adverse effects, including nephrotoxicity (3-5). In the kidney, cisplatin promotes primarily proximal tubular cell injury and death through several pathways, including apoptosis. The anti-tumor properties as well as the side effects of cisplatin are both dependent of its intracellular accumulation, which is mediated, at least in part, by membrane transporters. In the kidney, while organic cationic transporter type 2 (OCT2) and copper transporters (Ctrl and Ctr2) mediate the basolateral uptake of cisplatin in renal proximal tubular cells, other efflux transporters located at the apical cell membrane, including ATPase copper transporting alpha and beta (ATP7A, ATP7B) as well as Multidrug and toxin extrusion protein 1 and 2 (MATE1, MATE2), are involved in the secretion of cisplatin into the urine (6-7). Renal toxicity of cisplatin is cumulative and dose-dependent, leading to tubular lesions associated with a lower glomerular filtration rate (8-9). Cisplatin has been then reported to promote acute renal failure in up to 35% of patients, leading to cisplatin dose adjustment or even withdrawal, thereby adversely affecting patients' outcome (10-11).

In clinical practice, prevention of cisplatin-induced nephrotoxicity still largely relies on non-specific interventions, such as saline hydration or magnesium infusion (3, 11).

Therefore, there is an urgent medical need for strategies that alleviate cisplatin-induced nephrotoxicity, without interfering with the efficiency of cisplatin to control tumor growth.

Adenosine plays a major role in cellular and tissue homeostasis (13-15). Its physiological function relies on four G-protein coupled receptors, A1, A2A, A2B, and A3 (16-19). Adenosine is important for several aspects of the renal physiology (20-21) and adenosine and its receptors are engaged in various types of kidney injuries (22-26). In particular, the pharmacological blockade of A1 receptor using several antagonists, such as tonapofylline (27), 8-cyclopentyl-1,3-dipropylxanthine (28) or KW-3902 (29), has been reported to be protective against cisplatin nephrotoxicity in rodent models. Adenosine A2A receptor (A2AR) also control renal pathologies of various etiologies such as ischemia-reperfusion injury (30-31), fibrosis (25, 32), diabetic nephropathy (33) or glomerulonephritis (34). However, the role of A2AR remained unclear in the context of chemotherapeutic drug-induced toxicity.

### SUMMARY OF THE INVENTION

The present invention relates to a method for the treatment of chemotherapeutic drug-induced nephrotoxicity in a subject in need therefore comprising administering to the subject a therapeutically effective amount of a selective A_{2A} Adenosine Receptor (A2AR) antagonist. In particular, the present invention is defined by the claims.

### DETAILED DESCRIPTION OF THE INVENTION

Cisplatin is a potent chemotherapeutic drug, widely used in the treatment of various solid cancers. However, its clinical effectiveness is strongly limited by frequent severe adverse effects, such as nephrotoxicity. Therefore, there is an urgent medical need to identify novel strategies limiting chemotherapeutic drug-induced toxicity. Here, the inventors provide evidence that adenosine A2A receptor antagonist istradefylline (KW-6002) significantly protects from chemotherapeutic drug-induced nephrotoxicity in experimental models of acute and sub-chronic renal cisplatin intoxication. Importantly, they also demonstrate that the anti-tumoral properties of the chemotherapeutic drug are not altered by the antagonist in tumor-bearing mice and could even be potentiated at the molecular level.

Indeed, the inventors have surprisingly discovered that some nephrotoxic chemotherapeutic drugs, especially platin-containing chemotherapeutic drugs, are able to induce overexpression of A2A Adenosine Receptor in kidney. They have further discovered that administration of a selective A2A Adenosine Receptor antagonist is able to reduce nephrotoxicity induced by a chemotherapeutic drug, like a DNA-alkylating agent (particularly a platin-containing chemotherapeutic drug). Reduction or prevention of nephrotoxicity through blocking A2A Adenosine Receptors by binding them with a selective A2A Adenosine Receptors antagonist was unexpected. Altogether, the present results support the use of A2A Adenosine Receptors antagonist (e.g. istradefylline) as a new valuable preventive approach for the clinical management of patients undergoing chemotherapeutical drug (e.g. cisplatin) treatment.

The present invention relates to a method for the treatment of chemotherapeutic drug-induced nephrotoxicity in a subject in need therefore comprising administering to the subject a therapeutically effective amount of a selective A2A Adenosine Receptor (A2AR) antagonist.

The present invention relates to a method for the treatment of cisplatin-induced nephrotoxicity in a subject in need therefore comprising administering to the subject a therapeutically effective amount of a selective A2A Adenosine Receptor (A2AR) antagonist.

The present invention relates to a selective A2A Adenosine Receptor (A2AR) antagonist for use in the treatment of chemotherapeutic drug-induced nephrotoxicity in a subject in need thereof.

The present invention relates to a selective A2A Adenosine Receptor (A2AR) antagonist for use in the treatment of cisplatin-induced nephrotoxicity in a subject in need thereof.

In some embodiment, the present invention relates to istradefylline (KW-6002) or its derivatives for use in the treatment of chemotherapeutic drug-induced nephrotoxicity in a subject in need thereof. In some embodiment, the present invention relates to istradefylline (KW-6002) or its derivatives for use in the treatment of cisplatin-induced nephrotoxicity in a subject in need thereof.

As used herein, the term “subject” refers to a mammal, such as a rodent, a feline, a canine, and a primate. Particularly, the subject according to the invention is a human. Particularly, the subject according to the invention is an adult. Particularly, the subject according to the invention is a child. Particularly, the subject according to the invention is a teenager. Particularly, the subject according to the invention is a new born. As used herein, the term “subject” encompasses “patient”.

As used herein, the term “nephrotoxic” refers to the drug that can induce kidney injury and/or kidney inflammation when administrated in a pharmaceutically efficient dose or less and preferably when administrated in a pharmaceutically efficient dose. Particularly, a nephrotoxic drug means a drug able to increase plasmatic urea. The drug that can induce nephrotoxicity is called “chemotherapeutic drug induces nephrotoxicity”

In some embodiment, the chemotherapeutic drug induces nephrotoxicity is induced by cisplatin and is called “cisplatin-induced nephrotoxicity”.

As used herein, the term “A2A Adenosine Receptor receptor” (A2AR) also known as ADORA2A or RDC8 refers to an adenosine receptor, and also denotes the human gene encoding it. This protein is a member of the G protein-coupled receptor (GPCR) family which possess seven transmembrane alpha helices, as well as an extracellular N-terminus and an intracellular C-terminus. This protein plays an important role in many biological functions, such as heart rate and circulation, cerebral and renal blood flow, immune function, pain and sleep regulation. It has been implicated in pathophysiological states such as inflammatory diseases and disorders.

As used herein, the term “antagonist” or “inhibitor” refers to an agent (i.e. a molecule) which inhibits or blocks the activity of a receptor. For instance, an antagonist refers to a molecule which inhibits or blocks the activity of receptor.

As used herein, the term “biological activity” of A2A Adenosine Receptor refers to a nephrotoxicity associated with an inflammatory response and apoptosis, as exemplified by the increased mRNA expression of Il6 and Tnf.

Tests for determining the capacity of a compound to be a selective A2AR antagonist are well known to the person skilled in the art. In a preferred embodiment, the selective antagonist specifically binds to A2AR in a sufficient manner to inhibit the biological activity of A2AR. Binding to A2AR and inhibition of the biological activity of A2AR may be determined by any competing assays well known in the art. For example, the assay may consist in determining the ability of the agent to be tested as a selective A2AR antagonist to bind to A2AR. The binding ability is reflected by the Kd measurement. The term “KD”, as used herein, is intended to refer to the dissociation constant, which is obtained from the ratio of Kd to Ka (i.e. Kd/Ka) and is expressed as a molar concentration (M). KD values for binding biomolecules can be determined using methods well established in the art. In specific embodiments, a selective antagonist that “specifically binds to A_{2A}R” is intended to refer to an inhibitor that binds to human A_{2A}R polypeptide with a KD of 1 μM or less, 100 nM or less, 10 nM or less, or 3 nM or less. Then a competitive assay may be settled to determine the ability of the agent to inhibit biological activity of A_{2A}R. The functional assays may be envisaged such evaluating the ability a) decreased the nephrotoxicity associated with an inflammatory response and/or nephrotoxicity associated with apoptosis. The skilled in the art can easily determine whether a selective A_{2A}R antagonist neutralizes, blocks, inhibits, abrogates, reduces or interferes with a biological activity of A_{2A}R.

In some embodiment, the selective antagonist of the present invention (i.e. the selective A_{2A}R antagonists) the invention acts through direct interaction with the A_{2A} Adenosine Receptor.

As used herein, the term “A_{2A} Adenosine Receptor (A_{2A}R) antagonist” refers to an antagonist of the A_{2A} Adenosine Receptor.

In some embodiment, the selective A_{2A}R antagonists of the present invention can be used for reducing the acute nephrotoxicity induced by injection of a nephrotoxic chemotherapeutical drug and particularly a chemotherapeutical drug comprising cisplatin as an active compound, and particularly as the sole active compound. These selective antagonists can also be used to reduce the chronic nephrotoxicity induced by one or several (e.g. acute, subchronic model . . . ) administration of a nephrotoxic chemotherapeutical drug, as above-mentioned; the drug being active during several days after administration.

As used herein, the term “selective antagonist” is used as a shortcut for “selective A_{2A} Adenosine Receptors antagonist”. A selective antagonist according to the invention refers to an antagonist that specifically targets the receptor of A_{2A} Adenosine.

As used herein, the term “derivative” means that the derivative has been or may have been prepared from the underlying compound (i.e. a selective A_{2A}R antagonists) and shares the core structural components.

As used herein, the term “analog” refers a compound that is structurally and functionally related to another compound; compounds and analogs share high structural similarity and have similar biological functions.

In some embodiment, the selective A_{2A} Adenosine Receptor antagonist is preferably chosen among istradefylline (KW-6002) or its derivatives, MSX-3, SCH-58261 NIR178 (also known as PBF-509), ciforadenant, AB928, AZD4635, EOS100850,, Inupadenant (EOS-850), EXS21546, TT-10 and TT-53 and their pharmaceutically acceptable salts. Preferably, in all the embodiments, the selective A_{2A} Adenosine Receptor antagonist is preferably chosen among istradefylline (KW-6002) or its derivatives, MSX-3, SCH-58261, tozadenant (SYN 115) preladenant (SCH-420814), NIR178 (also known as PBF-509), ciforadenant, Etrumadenant (AB928), Imaradenant (AZD4635), EOS100850, Inupadenant (EOS-850), EXS21546, TT-10 and TT-53. Exemplary of substituted purine compounds thereof act as selective adenosine A_{2A} receptor (A_{2A}R) antagonists or its derivatives include also the compounds disclosed in the International Patent Application WO2021179074. For example, WO2021179074 includes the following compounds:

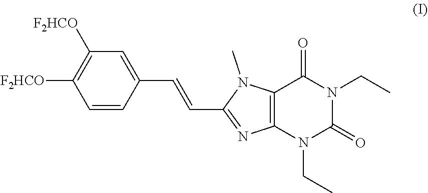 [SMILES: CCn1c(=O)c2c(nc(/C=C/c3ccc(OC(F)F)c(OC(F)F)c3)n2C)n(CC)c1=O]

or a pharmaceutically acceptable salt thereof

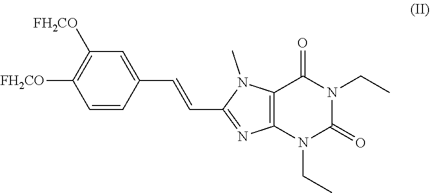

or a pharmaceutically acceptable salt

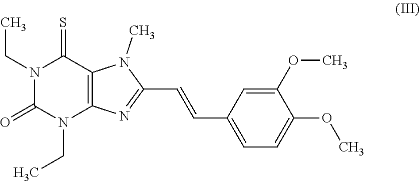 [SMILES: CCn1c(=S)c2c(nc(/C=C/c3ccc(OC)c(OC)c3)n2C)n(CC)c1=O]

or a pharmaceutically acceptable salt thereof.

These selective antagonists showed their efficiency in reducing nephrotoxicity induced by chemotherapeutic drug. They can be used alone, one after the other or in mixture of at least two antagonists.

In some embodiment, the present invention also relates to a selective A_{2A} Adenosine Receptor antagonist chosen among istradefylline (KW-6002) or its derivatives, MSX-3, SCH-58261 NIR178 (also known as PBF-509), ciforadenant, AB928, AZD4635, EOS100850, Inupadenant (EOS-850), EXS21546, TT-10 and TT-53 for use as a nephrotoxicity reducing agent in the treatment of cancer by at least one chemotherapeutic drug, said chemotherapeutic drug being able to induce nephrotoxicity when administrated to a subject and to induce overexpression of A_{2A} Adenosine Receptor in said subject, said chemotherapeutic drug being particularly chosen among pharmaceutical compositions comprising as an active compound a platin-containing compound and particularly a compound chosen among cisplatin, carboplatin, oxaliplatin and their pharmaceutically acceptable salts and more particularly being cisplatin.

As used herein, the term “nephrotoxicity reducing agent” relate to a pharmaceutically acceptable compound or composition able to reduce, when administered to a subject, at least one parameter chosen among plasmatic urea concentration, kidney histological score, creatinemia, proteinuria, NGAL relative expression and KIM-1 relative expression. The increase of the above-mentioned parameter is induced by administration of at least one chemotherapeutic agent. Cisplatin refers to cis-diamine-dichloroplatinum (II).

As used herein, the term “KW-6002” also known as Istradefylline means to 8-[(E)-2-(3,4-dimethoxyphenyl) vinyl]-1,3-diethyl-7-methyl-3,7-dihydro-1H-purine-2,6-dione. KW-6002 is having the following CAS number: 155270-99-8 and the following chemical formula:

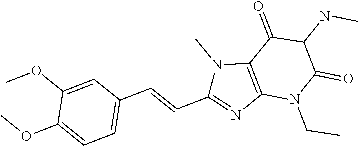

As used herein, the term “MSX-3” refers to 3,7-Dihydro-8-[(1E)-2-(3-methoxyphenyl) ethenyl]-7-methyl-3-[3-(phosphonooxy) propyl-1-(2-propynyl)-1H-purine-2,6-dione disodium salt hydrate. MSX-3 is having the following CAS number 261717-23-1 and the following chemical formula:

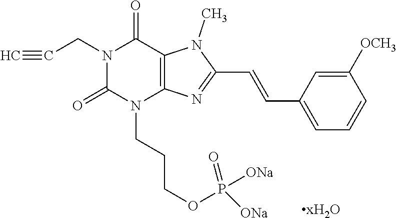

As used herein, the term “SCH-58261” refers to 2-(2-Furanyl)-7-(2-phenylethyl)-7H-pyrazolo[4,3-e][1,2,4]triazolo[1,5-c]pyrimidin-5-amine. SCH-58261 is having the following CAS number: 160098-96-4 and the following chemical formula:

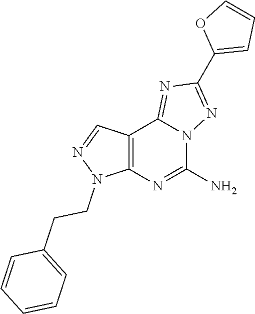 [SMILES: Nc1nc2c(cnn2CCc2ccccc2)c2nc(-c3ccco3)nn12]

As used herein, the term “Tozadenant” refers to 4-hydroxy-N-(4-methoxy-7-morpholin-4-yl-1,3-benzothiazol-2-yl)-4-methylpiperidine-1-carboxamide. Tozadenant is having the following CAS number: 870070-55-6 and the following chemical formula:

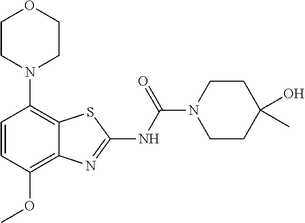 [SMILES: COc1ccc(N2CCOCC2)c2sc(NC(=O)N3CCC(C)(O)CC3)nc12]

As used herein, the term “Preladenant” refers to 2-(2-furanyl)-7-(2-(4-(4-(2-methoxyethoxy)phenyl)-1-piperazinyl)ethyl)-7H-pyrazolo (4,3-e) (1,2,4)triazolo (1,5-c)pyrimidine-5-amine. Preladenant is having the following CAS number: 377727-87-2 and the following chemical formula:

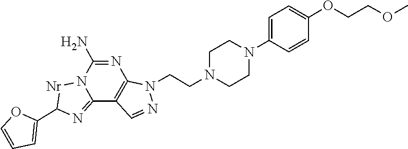

As used herein, the term “NIR178” also known as “Taminadenant” or “PBF-509” refers to 5-bromo-2,6-di(pyrazol-1-yl)pyrimidin-4-amine. NIR178 is having the following CAS number: 1337962-47-6 and the following chemical formula:

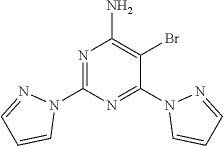 [SMILES: Nc1nc(-n2cccn2)nc(-n2cccn2)c1Br]

As used herein, the term “ciforadenant” refers to(S)-7-(5-methylfuran-2-yl)-3-((6-(((tetrahydrofuran-3-yl)oxy)methyl) pyridin-2-yl)methyl)-3H-[1,2,3]triazolo[4,5-d]pyrimidin-5-amine. Ciforadenanis is having the following CAS number: 1202402-40-1 and the following chemical formula:

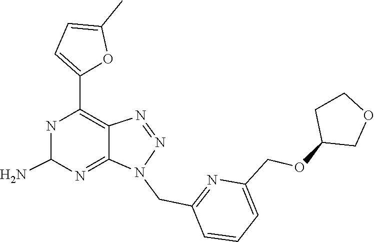

As used herein, the term “Etrumadenant” also known as “AB928” refers to 3-(2-amino-6-(1-((6-(2-hydroxypropan-2-yl) pyridin-2-yl)methyl)-1H-1,2,3-triazol-4-yl)pyrimidin-4-yl)-2-methylbenzonitrile. AB928 is having the following CAS number: 2239273-34-6 and the following chemical formula:

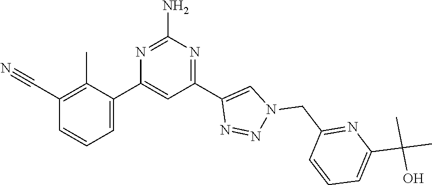 [SMILES: Cc1c(C#N)cccc1-c1cc(-c2cn(Cc3cccc(C(C)(C)O)n3)nn2)nc(N)n1]

As used herein, the term “Imaradenant” also knows as “AZD4635” refers to 6-(2-Chloro-6-methyl-4-pyridinyl)-5-(4-fluorophenyl)-1,2,4-triazin-3-amine. Imaradenant is having the following CAS number: 1321514-06-0 and the following chemical formula:

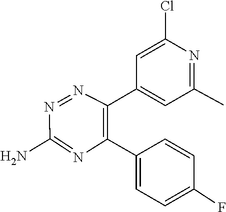 [SMILES: Cc1cc(-c2nnc(N)nc2-c2ccc(F)cc2)cc(Cl)n1]

As used herein, the term “EOS100850” refers to (+)-5-amino-3-{2-[4-(2,4-difluoro-5-{2-[(S)-methanesulfinyl]ethoxy}phenyl) piperazin-1-yl]ethyl}-8-(furan-2-yl) [1,3]thiazolo[5,4-e][1,2,4]triazolo[1,5-c]pyrimidin-2 (3H)-one hydrochloride. EOS100850 is having the following CAS number: 2411004-22-1 and the following chemical formula:

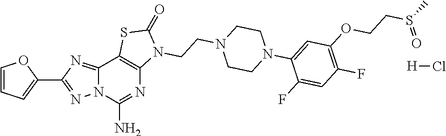

As used herein, the term “Inupadenant” also known as “EOS-850” refers to 7-amino-10-[2-[4-[2,4-difluoro-5-[2-[(S)-methylsulfinyl]ethoxy]phenyl]piperazin-1-yl]ethyl]-4-(furan-2-yl)-12-thia-3,5,6,8,10-pentazatricyclo[7.3.0.02,6]dodeca-1 (9),2,4,7-tetraen-11-one. Inupadenant is having the following CAS number: 2246607-08-7 and the following chemical formula:

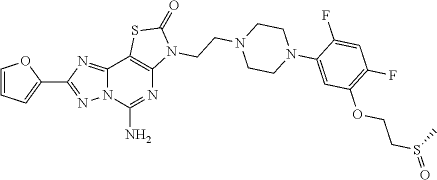 [SMILES: C[S@](=O)CCOc1cc(N2CCN(CCn3c(=O)sc4c3nc(N)n3nc(-c5ccco5)nc43)CC2)c(F)cc1F]

As used herein, the term “TT-10” refers to (2-(allylamino)-4-aminothiazol-5-yl) (5-fluorothiophen-2-yl) methanone. TT-10 is having the following CAS number: 2230640-94-3 and the following chemical formula:

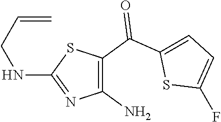 [SMILES: C=CCNc1nc(N)c(C(=O)c2ccc(F)s2)s1]

As used herein, the term “EXS21546” refers to a non-CNS penetrant A_{2A}R-selective antagonist from Exscientia Limited (ClinicalTrials Identifier: NCT04727138).

As used herein, the term “TT-53” refers to a selective Adenosine Receptor Antagonist from Tarus therapeutics.

The present invention also relates to the use of the selective A_{2A} Adenosine Receptor antagonist with combination with chemotherapeutic. In some embodiment, the therapeutic use of chemotherapeutic drug induces the occurrence of nephrotoxicity.

As used herein, the term “chemotherapy” has its general meaning in the art and refers to the treatment that consists in administering to the patient a chemotherapeutic agent. Chemotherapeutic agents include, but are not limited to alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall; dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxy doxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-1 1); topoisomerase inhibitor RFS 2000; difluoromethylomithine (DMFO); retinoids such as retinoic acid; capecitabine; and pharmaceutically acceptable salts, acids or derivatives of any of the above.

As used herein, the term “chemotherapeutical drug” refers to any pharmaceutically acceptable composition being therapeutically or pharmaceutically efficient in cancer treatment. A chemotherapeutical drug can be a DNA-alkylating drug, for example.

As used herein, the term “nephrotoxic chemotherapeutic drug” refers to any pharmaceutically acceptable composition being therapeutically or pharmaceutically efficient in cancer treatment that can induce kidney injury and/or kidney inflammation when administrated in a pharmaceutically efficient dose or less and preferably when administrated in a pharmaceutically efficient dose.

In some embodiment, the nephrotoxic chemotherapeutic drug is chosen among DNA-alkylating agents, particularly among platin-containing chemotherapeutic drugs and more particularly among chemotherapeutic drugs comprising an active compound chosen among cisplatin, carboplatin, oxaliplatin and the pharmaceutically acceptable salts thereof.

As used herein, the term “Cisplatin” refers to cis-diamminedichloroplatinum (II) and is a drug used in chemotherapy mainly because it blocks DNA synthesis and induces apoptosis through a mechanism of action via p53. Cisplatin is having the following CAS number: 15663-27-1 and the following chemical formula:

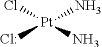

As used herein, the term “Oxaliplatin” refers to to [(1R,2R)-cyclohexane-1,2-diamine](ethanedioato-O,O′) platinum (II). Oxaliplatin is having the following CAS number: 63121-00-6 and the following chemical formula:

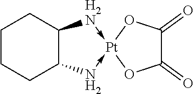

As used herein, the term “Carboplatin” refers to cis-diamine (cyclobutane-1,1-dicarboxylate-O,O′) platinum (II). Carboplatin is having the following CAS number: 41575-94-4 and the following chemical formula:

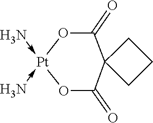

As used herein, the terms “able to induce overexpression of A_{2A} Adenosine Receptors” means that when the drug is administrated in a given amount, in a pharmaceutically effective amount or dosage, for example, the relative expression of A_{2A} Adenosine Receptors is over 1-fold and more particularly over 1.1-fold.

The present invention relates a new nephrotoxicity-reducing agent that can be used in cancer treatment by administration of administration of a selective A_{2A} Adenosine Receptors antagonist and/or at least one nephrotoxic chemotherapeutic drug.

In some embodiment, the present invention also relates a method for the treatment of cancer in a subject in need thereof comprising a therapeutically effective amount of a combination of a selective A_{2A} Adenosine Receptor antagonist and a chemotherapeutic drug.

In some embodiment, the present invention also relates a method for the treatment of cancer in a subject in need thereof comprising a therapeutically effective amount of a combination of KW-6002 or its derivatives and cisplatin.

The inventors show that the selective A_{2A} Adenosine Receptor antagonist improves the anti-tumoral effect of the cisplatin.

In some embodiment, the present invention also relates a combination of a selective A_{2A} Adenosine Receptor antagonist and a chemotherapeutic drug for use as an anti-tumoral treatment in a subject in need thereof.

In some embodiment, the present invention also relates a combination of KW-6002 or its derivatives and cisplatin method for use as an anti-tumoral treatment in a subject in need thereof.

As used herein the term “cancer” has its general meaning in the art and refers to a group of diseases involving abnormal cell growth with the potential to invade or spread to other parts of the body. The term “cancer” further encompasses both primary and metastatic cancers. Examples of cancers that may treated by methods and compositions of the invention include, but are not limited to, cancer cells from the bladder, blood, bone, bone marrow, brain, breast, colon, esophagus, gastrointestinal, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; non encapsulating sclerosing carcinoma; adrenal cortical carcinoma; endometroid carcinoma; skin appendage carcinoma; apocrine adenocarcinoma; sebaceous adenocarcinoma; ceruminous; adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; Sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malign melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brennertumor, malignant; phyllodestumor, malignant; synovial sarcoma; mesothelioma, malignant; dysgerminoma; embryonal carcinoma; teratoma, malignant; strumaovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor ewing's sarcoma; odontogenic tumor, malignant; ameloblasticodontosarcoma; ameloblastoma, malignant; ameloblasticfibrosarcoma; pinealoma, malignant; chordoma; glioma, malignant; ependymoma; ependymoma; protoplasmic astrocytoma; fibrillary astrocytoma; astroblastoma; medulloblastoma, glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocyticleukemia; mast cell leukemia; megakaryoblasticleukemia; myeloid sarcoma; and hairy cell leukemia.

As used herein the terms “administering” or “administration” refer to the act of injecting or otherwise physically delivering a substance as it exists outside the body (e.g. a selective A_{2A} Adenosine Receptor (A_{2A}R) antagonist) into the subject, such as by mucosal, intradermal, intravenous, subcutaneous, intramuscular delivery and/or any other method of physical delivery described herein or known in the art. When a disease, or a symptom thereof, is being treated, administration of the substance typically occurs after the onset of the disease or symptoms thereof. When a disease or symptoms thereof, are being prevented, administration of the substance typically occurs before the onset of the disease or symptoms thereof.

A “therapeutically effective amount” refers to an amount effective, at dosages and for periods of time necessary, to achieve a desired therapeutic result. A therapeutically effective amount of drug may vary according to factors such as the disease state, age, sex, and weight of the individual, and the ability of drug to elicit a desired response in the individual. A therapeutically effective amount is also one in which any toxic or detrimental effects of the antibody or antibody portion are outweighed by the therapeutically beneficial effects. The efficient dosages and dosage regimens for drug depend on the disease or condition to be treated and may be determined by the persons skilled in the art. A physician having ordinary skill in the art may readily determine and prescribe the effective amount of the pharmaceutical composition required. For example, the physician could start doses of drug employed in the pharmaceutical composition at levels lower than that required in order to achieve the desired therapeutic effect and gradually increase the dosage until the desired effect is achieved. In general, a suitable dose of a composition of the present invention will be that amount of the compound which is the lowest dose effective to produce a therapeutic effect according to a particular dosage regimen. Such an effective dose will generally depend upon the factors described above. For example, a therapeutically effective amount for therapeutic use may be measured by its ability to stabilize the progression of disease. One of ordinary skill in the art would be able to determine such amounts based on such factors as the subject's size, the severity of the subject's symptoms, and the particular composition or route of administration selected. An exemplary, non-limiting range for a therapeutically effective amount of drug is about 0.1-100 mg/kg, such as about 0.1-50 mg/kg, for example about 0.1-20 mg/kg, such as about 0.1-10 mg/kg, for instance about 0.5, about such as 0.3, about 1, about 3 mg/kg, about 5 mg/kg or about 8 mg/kg. Administration may e.g. be intravenous, intramuscular, intraperitoneal, or subcutaneous, and for instance administered proximal to the site of the target. Dosage regimens in the above methods of treatment and uses are adjusted to provide the optimum desired response (e.g., a therapeutic response). For example, a single bolus may be administered, several divided doses may be administered over time or the dose may be proportionally reduced or increased as indicated by the exigencies of the therapeutic situation. In some embodiments, the efficacy of the treatment is monitored during the therapy, e.g. at predefined points in time. As non-limiting examples, treatment according to the present invention may be provided as a daily dosage of the agents of the present invention in an amount of about 0.1-100 mg/kg, such as 0.2, 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, per days, on at least one of days 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of weeks 1, 2, 3, 4, 5, 6, 7, 8, 9, 10, 11, 12, 13, 14, 15, 16, 17, 18, 19 or 20 after initiation of treatment, or any combination thereof, using single or divided doses every 24, 12, 8, 6, 4, or 2 hours, or any combination thereof.

In some embodiment the therapeutically effective amount of the selective A_{2A} Adenosine Receptor antagonist, can be equal to 10 mg or more and equal to 350 mg or less and particularly equal to 20 mg, 25 mg, 30 mg, 35 mg, 40 mg, 50 mg, 60 mg, 70 mg, 80 mg, 90 mg, 100 mg, 110 mg or 120 mg, 160 mg, 180 mg, 210 mg or 240 mg. In a particular embodiment, when the selective A_{2A} Adenosine Receptor antagonist is KW-6002 or its derivatives, its therapeutically effective amount is preferably under a maximum value, this value can be equal to or more than 60 mg to 230 mg and more preferably equal to 70 mg or more and equal to 210 mg or less. As herein after explained, when the amount of this particular selective antagonist is over a threshold, it has no more activity as a nephrotoxicity reducing agent, and particularly in reducing nephrotoxicity induced by chemotherapeutic drug (e.g. cisplatin).

In some embodiment, the therapeutically effective amount of the chemotherapeutical drug can be equal to 80 mg or more and equal to 1400 mg or less and particularly can be equal to 140 mg, 147 mg, 175 mg, 630 mg, 692 mg and 700 mg. Values particularly suitable when the chemotherapeutic drug comprises as an active compound chemotherapeutic drug (e.g. cisplatin) and particularly when said drug comprises as the sole active compound chemotherapeutic drug (e.g. cisplatin) are 140 mg, 700 mg and 1400 mg.

As used herein, the term “combination” is intended to refer to all forms of administration that provide a first drug together with a further (second, third . . . ) drug. The drugs may be administered simultaneously, separately or sequentially and in any order. According to the invention, the drug is administered to the subject using any suitable method that enables the drug to reach the chondrocytes of the bone growth plate. In some embodiments, the drug administered to the subject systemically (i.e. via systemic administration). Thus, in some embodiments, the drug is administered to the subject such that it enters the circulatory system and is distributed throughout the body. In some embodiments, the drug is administered to the subject by local administration, for example by local administration to the growing bone.

As used herein, the terms “combined treatment”, “combined therapy” or “therapy combination” refer to a treatment that uses more than one medication. The combined therapy may be dual therapy or bi-therapy.

As used herein, the term “administration simultaneously” refers to administration of 2 active ingredients by the same route and at the same time or at substantially the same time. The term “administration separately” refers to an administration of 2 active ingredients at the same time or at substantially the same time by different routes. The term “administration sequentially” refers to an administration of 2 active ingredients at different times, the administration route being identical or different.

In some embodiment, the selective A_{2A} Adenosine Receptor antagonist is to be administrated before administration of said chemotherapeutic drug. and preferably, at least one hour before said chemotherapeutical drug administration.

In some embodiment, the chemotherapeutic drug can be administrated in one administration. In this case, the selective antagonist can be administrated before the chemotherapeutic drug administration, preferably at least one hour before. It can also be administrated before and after the chemotherapeutic drug administration. For example, it can be administrated at least one time, the day after the chemotherapeutic drug administration and for example, once a day over one day, two days, five days or 8 days after the chemotherapeutic drug administration. When the chemotherapeutic drug and particularly when the chemotherapeutic drug comprises as an active compound cisplatin and preferably cisplatin as the sole anticancer active compound and as the sole active compound, is administrated several times, once a day over several days, for example (one a day, over a week, for example), the selective antagonist is preferably administrated before each administration of the chemotherapeutic drug, and for example, at least one haour before. The selective antagonist can also be administrated once a day after the last administration of said chemotherapeutic drug and particularly over at least one day, or two days after the last administration of said chemotherapeutic drug and for example, over 5 days after the last administration of said chemotherapeutic drug.

The present invention also relates to a therapeutically effective amount of a combination of a selective A_{2A} Adenosine Receptor (A_{2A}R) antagonist and chemotherapeutic drug for use in the treatment of chemotherapeutic drug-induced nephrotoxicity.

The present invention also relates to a therapeutically effective amount of a combination of a selective A_{2A} Adenosine Receptor (A_{2A}R) antagonist and chemotherapeutic drug for use in the treatment of cisplatin-induced nephrotoxicity.

In a particular embodiment, the invention relates to a therapeutically effective amount of a combination of KW-6002 and cisplatin for use in the treatment of chemotherapeutic drug-induced nephrotoxicity.

In a particular embodiment, the invention relates to a therapeutically effective amount of a combination of KW-6002 and cisplatin for use in the treatment of chemotherapeutic drug-induced nephrotoxicity.

In a particular embodiment, the invention relates to a therapeutically effective amount of a combination of KW-6002 and cisplatin for use in the treatment of cisplatin-induced nephrotoxicity.

The present invention also relates to a i) selective A_{2A} Adenosine Receptor (A_{2A}R) antagonist and ii) chemotherapeutic drug for simultaneous, separate or sequential use in the treatment of chemotherapeutic drug-induced nephrotoxicity.

The present invention also relates to a i) selective A_{2A} Adenosine Receptor (A_{2A}R) antagonist and ii) chemotherapeutic drug for simultaneous, separate or sequential use in the treatment of cisplatin-induced nephrotoxicity.

In a particular embodiment, the invention relates to a i) KW-6002 and ii) cisplatin for simultaneous, separate or sequential use in the treatment of chemotherapeutic drug-induced nephrotoxicity.

In a particular embodiment, the invention relates to a i) KW-6002 and ii) cisplatin for simultaneous, separate or sequential use in the treatment of cisplatin-induced nephrotoxicity.

The present invention also relates to a pharmaceutical composition comprising a therapeutically effective amount of a selective A_{2A} Adenosine Receptor (A_{2A}R) antagonist and a chemotherapeutic drug. The pharmaceutical composition according to the invention wherein the selective A_{2A} Adenosine Receptor (A_{2A}R) antagonist is KW-6002 and wherein the chemotherapeutic drug is cisplatin.

More particularly, the pharmaceutical composition according to the invention is suitable for treating chemotherapeutic drug-induced nephrotoxicity.

More particularly, the pharmaceutical composition according to the invention is suitable for treating cisplatin-induced nephrotoxicity.

The selective A_{2A} Adenosine Receptor (A_{2A}R) antagonist and/or the chemotherapeutic drug described above may be combined with pharmaceutically acceptable excipients, and optionally sustained-release matrices, such as biodegradable polymers, to form pharmaceutical compositions. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. The pharmaceutical compositions of the present invention for oral, sublingual, subcutaneous, intramuscular, intravenous, transdermal, parenteral, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal, parenteral and intranasal administration forms and rectal administration forms. Typically, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The polypeptide (or nucleic acid encoding thereof) can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active polypeptides in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

The invention will be further illustrated by the following figures and examples. However, these examples and figures should not be interpreted in any way as limiting the scope of the present invention.

### FIGURES

FIG. 1. Schematic representation of the different animal procedures. (A) Acute cisplatin-induced kidney injury. C57BL6/J 8-weeks old male mice were administered an intra-peritoneal single injection of 10 mg/kg cisplatin (cis) and were sacrificed (E) three days (D) post-injection. (B) Sub-chronic cisplatin-induced kidney injury. C57BL6/J 8-weeks old male mice were intraperitoneally injected daily with cisplatin (3 mg/kg) for 6 days and were sacrificed 72 h after the last injection. (C) KW-6002 administration schedule in the acute model. C57BL6/J 8-weeks old male mice were randomized to 4 groups: Vehicle, KW-6002, Cisplatin or KW6002/Cisplatin. Cisplatin administration was performed as indicated in A. The first administration of KW6002 (3 mg/kg) was performed one day prior cisplatin treatment and daily until the sacrifice. (D) KW-6002 administration schedule in the sub-chronic model. C57BL6/J 8-weeks old male mice were randomized to 4 groups: Vehicle, KW-6002, Cisplatin or KW-6002/Cisplatin. Cisplatin administration was performed as indicated in B. The first administration of KW-6002 (3 mg/kg) was performed five days prior cisplatin treatment and daily until the sacrifice. (E) Effects of KW-6002 in the LLC syngeneic tumor mouse model treated with sub-chronic cisplatin. KW-6002 and cisplatin administration were as performed in D. LLC cells (10 millions) in PBS:matrigel (1:1, for a total volume of 100 μL) were subcutaneously injected in the right flank of all animals. When tumor volume reached 100 mm³, mice were randomized into 4 groups: Vehicle, KW-6002, Cisplatin or KW-6002/Cisplatin.

FIG. 2. KW-6002 protects from cisplatin-induced kidney injury in acute (A-F) and sub-chronic (G-L) models. (A, G) BUN quantification. (B, H) Histological score of renal injury. Gene expression of the renal injury markers NGAL (C, I) and KIM-1 (D, J). Gene expression of the inflammatory markers Tnfα (E, K) and I16 (F, L) . . . . Results are expressed as mean±SEM. ** p<0.01, *** p<0.001 vs. Vehicle; °°p<0.01, 00° p<0.001 vs. Cisplatin (n=6-17 animals/group; One-Way ANOVA followed by Tukey's post-hoc test).

FIG. 3. KW-6002 protects nephrotoxicity without attenuating the anti-tumoral properties of cisplatin in a syngeneic in vivo mouse model. Absolute tumor size in the different groups of animals. Results are expressed as mean±SEM. *** p<0.001 vs. Vehicle; 000p<0.001 vs. Cisplatin (n=13-25 animals/group; One-Way ANOVA followed by Tukey's post-hoc test). ** p<0.01 (n=6-10 animals per group; Two-Way ANOVA).

FIG. 4. KW-6002 limits cisplatin accumulation in kidney but not in tumors. (A) Platinum quantification (n=12-20/group) and relative expression of influx transporters (CTR1 and OCT2) and efflux transporters (ATP7B, MATE1, MATE2 and ABCC2; n=10-12/group) in kidney. (B) Platinum quantification (n=12-20/group) and relative expression of influx transporters (CTR1) and efflux transporters (ATP7B, MATE1, and ABCC2; n=5-6/group) in tumors. Results are expressed as mean±SEM. * p<0.05, *** p<0.001 vs. Vehicle; °p<0.05, °°° p<0.001 vs. Cisplatin (One-Way ANOVA followed by Tukey's post-hoc test). (C) Active efflux in RPTEC/hTERT cells. * P<0.05 versus vehicle; ° P<0.05 versus cisplatin; 1-way ANOVA followed by Tukey's post hoc test. Results are the mean±SEM (n=3 independent experiments)

FIG. 5. KW6002 protects from cisplatin-induced kidney injury in a cumulative model of cisplatin toxicity. BUN quantification. Data are mean±SEM. * p<0.05, *** p<0.001 vs. Vehicle; p<0.05, °° p<0.01, °°° p<0.001 vs. cisplatin (n=6 animals/group; Two-Way ANOVA followed by a Tukey's post-hoc test).

FIG. 6. KW6002 alleviates the cisplatin-induced nephrotoxicity associated with apoptosis, lipid metabolism, and oxidative stress. (A) Tail moment assessed by comet assay; 50 cells/condition were analyzed. (B and C) KW6002 attenuates cisplatin-induced renal lipid accumulation, assessed by Red Oil staining. (B) Representative quantification (n=5 randomly chosen fields/staining). *** P<0.001 versus vehicle; °°P<0.01 versus cisplatin; 1-way ANOVA followed by Tukey's post hoc test (n=3 independent experiments). (C) Representative quantification (n=8 randomly chosen fields/section) Results are the mean+SEM. * P<0.05 versus vehicle; ° P<0.05 versus cisplatin; 1-way ANOVA followed by Tukey's post hoc test (n=3 mice/group). (D and E) KW6002 attenuated cisplatin-induced oxidative stress in the subchronic cisplatin mouse model. Gene expression of Nrf2 (D) and HO1 (E). ** P<0.01 and *** P<0.001 versus vehicle; ° P<0.05 and °°P<0.01 versus cisplatin; 1-way ANOVA followed by Tukey's post hoc test. Results are the mean±SEM (n=6-9 mice/group). (F-H) KW6002 attenuated cisplatin-induced oxidative stress in RPTEC/hTERT1 cells. (F and G) Gene expression of Nrf2 (F) and HO1 (G). (H) Catalase activity. * P<0.05 and ** P<0.01 versus vehicle; °P<0.05 versus cisplatin; 1-way ANOVA followed by Tukey's post hoc test. Results are the mean±SEM (n=3 independent experiments).

FIG. 7. KW6002 does not interfere with the antitumoral effect of cisplatin. (A and B) LLC1 cells were exposed to 2 μM cisplatin with or without 10 Nm KW6002 for 24 hours (A) or 6 hours (B). Caspase 3/-7 activity (n=3 independent experiments) (A) and the number of γH2AX nuclear foci (n=36-63 nuclei/group) (B) were determined. Results are the mean±SEM. *** P<0.001 versus vehicle; °°P<0.01 and 00° P<0.001 versus cisplatin; 1-way ANOVA followed by Tukey's post hoc test. (C, D, G, and H) H1975 cells were exposed to 20 μM cisplatin with or without 10 nM KW6002 for 24 hours. Quantification of caspase 3 (C), PARP (D), cyclin D1 (G), and PCNA (H). * P<0.05 and ** P<0.01 versus vehicle; °°P<0.01 versus cisplatin; 1-way ANOVA followed by Tukey's post hoc test. Results are expressed as the mean±SEM (n=3 independent experiments). (E) Tail moment was assessed by comet assay. A total of 50 cells per condition were analyzed. *** P<0.001; 1-way ANOVA followed by Tukey's post hoc test. (F) H1975 cells were exposed to 50 μM cisplatin with or without 10 nM KW6002 for 6 hours, and the number of yH2AX nuclear foci was counted. Results are expressed as the mean±SEM. * P<0.05 versus vehicle; ° P<0.01 versus cisplatin; 1-way ANOVA followed by Tukey's post hoc test (n=30-42 nuclei/condition). (I) KW6002 increased yH2AX expression in tumors from mice of the LLC1 syngeneic model. Quantification of nuclear yH2AX expression. The percentage of positive cells was calculated using 5 randomly chosen fields per staining. * P<0.05 versus vehicle; ° P<0.01 versus cisplatin; 1-way ANOVA followed by Tukey's post hoc test (n=5/group).

FIG. 8. KW6002 prevents nephrotoxicity and neurotoxicity without attenuating the antitumoral properties of cisplatin in the mEERL syngeneic in vivo mouse model. (A-C) KW6002 alleviated cisplatin-induced nephrotoxicity as estimated by mRNA levels of KIM1 (A) and the inflammatory markers Tnfα and I16 (B and C). * P<0.05 and *** P<0.001 versus vehicle; °P<0.01 and °°P<0.01 versus cisplatin; 1-way ANOVA (n=5-6/group). (D) Mechanical sensitivity measured by von Frey hairs in mice in response to cisplatin and/or KW6002. The arrow represents cisplatin and/or PBS injection. Data are the mean±SEM. *** P<0.001 versus vehicle; °°° P<0.001 versus cisplatin; 2-way ANOVA (n=5/group). (E) Absolute tumor sizes in animals in the different groups. Results indicate the mean±SEM. ***P<0.001 versus vehicle; ° P<0.05 versus cisplatin; 2-way ANOVA (n=5-6 animals/group).

FIG. 9. MSX-3 protects from cisplatin-induced kidney injury a mouse model. (A) BUN quantification. (B) Gene expression of the inflammatory marker Tnfa. Results are expressed as mean±SEM. ** p<0.01, *** p<0.001 vs. Vehicle; °°p<0.01, °°° p<0.001 vs. Cisplatin (n=6-17 animals/group; One-Way ANOVA followed by Tukey's post-hoc test).

### EXAMPLE

### Material & Methods

### Animals and Treatments.

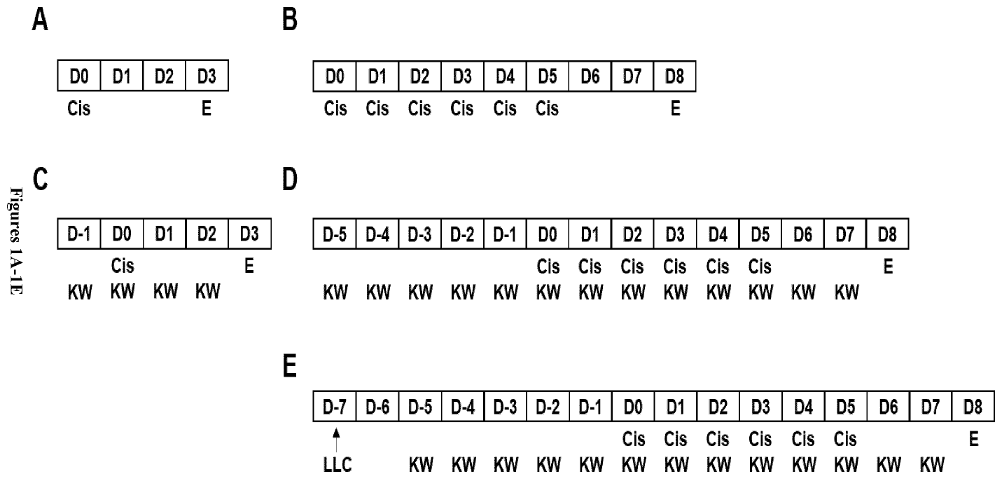

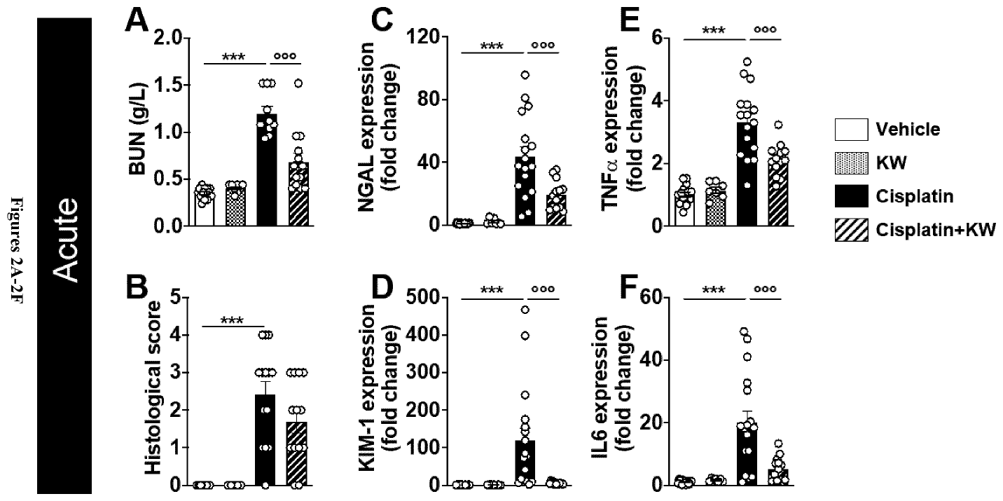

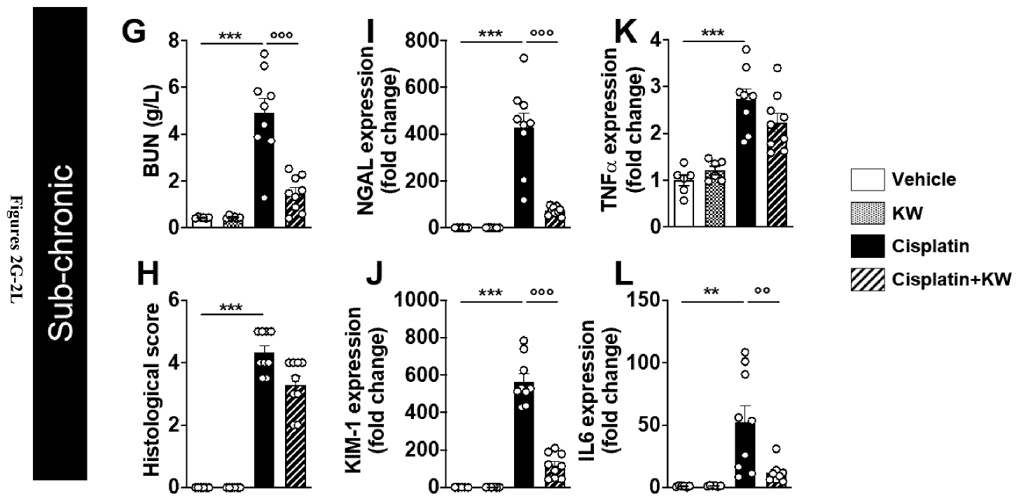

Animal experiments were adapted from 63-64. Animal procedures were performed in 8 to 10 weeks old male C57B16/J mice (Janvier Labs). Mice were fed a laboratory standard diet with water and food ad libitum and were kept under constant environmental conditions with a 12-hour light-dark cycle. Istradefylline (KW-6002; Tocris) was dissolved in a carrier solution consisting in 15% DMSO, 15% cremophor (Sigma), 70% saline solution (vehicle). Cisplatin (Accord Healthcare) was dissolved in saline solution. Acute cisplatin nephrotoxicity was induced following a single intra-peritoneal (i.p.) injection (day 0) of 10 mg/kg cisplatin. Three days after this single injection, animals were sacrificed by cervical dislocation (FIG. 1A). When KW-6002 (3 mg/kg) was tested against acute cisplatin toxicity, the drug was administered daily i.p. from day-1 to day 2 (FIG. 1C). Toxicity of sub-chronic cisplatin was evaluated following six daily i.p. injections of cisplatin (3 mg/kg) starting at day 0 and mice were sacrificed 72h after the last injection of cisplatin (day 8; FIG. 1B). When KW-6002 was tested against sub-chronic cisplatin toxicity, the drug was administered daily i.p. from day-5 to day 7 (FIG. 1D).

### LLC1 In Vivo Tumor Model.

LLC1 cells (Lewis Lung Cancer cells, ATCC® CRL-1642™) were cultured in DMEM with 10% fetal calf serum and penicillin-streptomycin. LLC1 cells (107) in PBS: matrigel (1:1, for a total volume of 100 μL) were subcutaneously injected in the right flank of animals. Tumors were measured twice a week with calipers and their volumes were estimated using the following equation: ½ (length×width2). When tumor volume reached 100 mm³, mice were randomly ascribed to the four experimental groups (Vehicle, KW-6002, Cisplatin and Cisplatin/KW-6002) as indicated in FIG. 1E.

mEERL In Vivo Tumor Model.

We used a validated murine model of HPV+ oropharyngeal squamous cell carcinoma as previously described (noncommercial) (77). This model consists of oropharyngeal epithelial cells from C57Bl/6 male mice that stably express the HPV16 viral oncogenes E6 and E7, H-Ras, and luciferase (mEERL cells). mEERL cells were grown in a T75 flask until confluent, after which cells were trypsinized and harvested, washed 3 times with sterile PBS, and resuspended in 1 mL sterile PBS to the appropriate concentration. Mice were injected s.c. into the right flank with 20 μL solution containing either 1,000,000 mEERL cells or PBS (vehicle). The day of mEERL cell injection is indicated as day −14. Tumor volume was monitored using Vernier digital calipers. When the tumor volume reached 100 mm³, the mice were randomly ascribed to 1 of the 3 experimental groups (vehicle; cisplatin; or cisplatin plus KW6002).

### Sample Collection.

Prior sacrifice by cervical dislocation, retro-orbital blood samples were collected in heparinized tubes and centrifuged for 10 min at 2000 rpm at room temperature. Renal function was assessed by Blood urea nitrogen (BUN) measurement using a AU480 Chemistry Analyzer (Beckman Coulter). At the time of sacrifice, kidneys or LLC1 tumors were harvested and stored in either “RNA later” solution (Thermo Fisher), 4% neutral buffered formalin or snap-frozen in liquid nitrogen.

### Renal Histological Analysis.

Formalin-fixed and paraffin-embedded sections (3 μm thick) were stained with Hematoxylin and Eosin (Sigma Aldrich) or Periodic Acid-Schiff (Sigma-Aldrich). Slices were scored by a nephropathologist in a blinded manner. A kidney injury score grading scale from 0 to 5 was used to assess the severity of the injury as follow: 0=no lesions, 1=minimal injury characterized by occurrence of necrosis and debris; 2=mild injury with single cell necrosis, pyknotic cells, and apoptosis; 3=moderate injury characterized by tubular distension, vacuolation, and some cellular debris, 4=severe injury with occasional hyaline casts observed, patchy epithelial necrosis in all segments, loss of epithelial lining; and 5=very severe injury characterized by extensive tubular epithelial necrosis in all segments, loss of epithelial layer from many tubules widespread intraluminal cellular debris, and frequent hyaline casts particularly prominent in the medullary region (67).

### Cell Culture.

Renal proximal tubular epithelial cells (RPTEC) immortalized with a pLXSN-hTERT retroviral vector (ATCC® CRL-4031™) is a relevant in vitro model to evaluate cisplatin deleterious effects (68-70). Cells were cultured in DMEM/F12 medium (Dulbecco's modified Eagle's medium and Ham's F12 medium, Thermo Fisher) supplemented with 1% penicillin/streptomycin, 5 pmol/L triiodo-L-thyronine, 10 ng/ml recombinant human epidermal growth factor, 3.5 μg/mL ascorbic acid, 5.0 μg/mL human transferrin, 5.0 μg/mL insulin, 25 ng/mL prostaglandin E1, 25 ng/ml hydrocortisone, 8.65 ng/mL sodium selenite, 0.1 mg/mL G418 and 1.2 g/L sodium bicarbonate (Sigma). Lewis lung carcinoma mouse LLC1 cells were cultured in DMEM glutamax (Thermo Fisher) containing 10% fetal calf serum and 1% penicillin/streptomycin. Cells were cultured at 37° C. in a humidified atmosphere of 5% CO2.

### Cell Viability Assay.

RPTEC/hTERT cells were cultured in 96-well plates (40,000 cells/well) and exposed to cisplatin (50 μM) without or with KW-6002 (0.5-12.8 μM) for 48 hours. Viability was assessed using the CellTiter-Glo® Luminescent Cell Viability Assay (Promega) according to manufacturer's recommendations.

### Caspase 3/7 Activity.

RPTEC cells were cultured in 96-well plates (40,000 cells/well) and exposed to cisplatin (50 μM) without or with KW-6002 for 48 hours. LLC1 cells were cultured in 96-well plates (10,000 cells/well) and after 24 hours, exposed to 2 μM of cisplatin without or with 10 nM of KW-6002 for 24 hours. Apoptosis was assessed in RPTEC/hTERT and LLC1 cell lysates using the caspase-Glo 3/7 assay (Promega) according to manufacturer's recommendations.

### Catalase Activity.

RPTEC/hTERT1 cells were cultured in 6-well plates (250,000 cells/well) and exposed for 48 hours to cisplatin (50 μM) with or without KW6002 (25 μM). Catalase activity was assessed using the Catalase Colorimetric Activity Kit (Thermo Fisher Scientific) according to the manufacturer's instructions.

### Cell Efflux.

The basic cell efflux function was assessed using an EFLUXX-ID Green Multidrug Resistance Assay Kit (ENZO Life Sciences). Briefly, 2.5×105 cells/condition were collected, washed with PBS, and incubated with the EFLUXX-ID Green Detection Reagent for 30 minutes at 37° C., and then efflux was measured immediately by flow cytometry (CytoFLEX LX, Beckman Coulter). All experiments were performed in triplicate, with the measurement of 10,000 individual cells. Data were analyzed using Kaluza Analysis Software (Beckman Coulter).

### Comet Assay.

Treated cells were suspended (60,000 cells/mL) in low-melt agarose (1613111, Bio-Rad) 0.5% in PBS at 42° C. The suspension was then immediately spread on a comet slide (4250-200-03, R&D Systems). Agarose was allowed to cool down for 20 minutes at 4° C. Then, cell membranes were permeabilized with a lysis solution (2.5M NaCl, 100 mM EDTA, 10 mM Tris-HCl, 1% Triton X-100 [pH 10]) at 4° C. for 1 hour. Slides were then equilibrated for 20 minutes in electrophoresis buffer (pH 12.3:2 mM EDTA, pH adjusted to 12.3 with NaOH) at 4° C. Then, an electrophoresis field of 2.06 V/cm (98 V and approximatively 176 mA in an electrophoretic system where electrodes are 47.5 cm apart) was applied for 5 minutes at 4° C. for RPTEC/hTERT1 cells, or for 3 minutes 30 seconds for H1975 cells. The electrophoretic migration was stopped by neutralizing the pH in a bath of cold water for 10 minutes. DNA was stained with SYBR Green (S7563, Invitrogen, Thermo Fisher Scientific) for 20 minutes at room temperature, according to the manufacturer's recommendation. The slides were photographed under an Axio Imager Z1 Apotome microscope (Zeiss). The images were analyzed using an ImageJ in-home macro, in which the head (the nucleus) and tail (the DNA that migrated) of the comet were delimited to get the fluorescence intensity of the head, the fluorescence intensity of the tail, and the length of the tail. The calculation of tail moments was done using the following formula: (length of the comet tail×fluorescence intensity of the tail)/total fluorescence intensity (head+tail).

### Red Oil Staining.

10 μm frozen kidney mouse sections were fixed with ethanol (60%) then incubated for 15 min with Oil Red O Solution (Fisher Biotech) dissolved in isopropanol. After several washes with ddH2O, samples were incubated 3 min with hematoxylin. Lipid droplets are stained in red whereas nuclei appeared blue. RPTEC/hTERT cells were grown on coverslips in 24 well plates (75,000 cells/well) and exposed to cisplatin (50 μM) without or with 25 μM KW-6002 for 48 hours. RPTEC/hTERT cells were fixed with ethanol (60%) then incubated for 15 min with Oil Red O Solution. The cells were washed three times with ddH2O and incubated 3 min with hematoxylin. Coverslips were rinsed with H2O before mounting on microscope slides using glycerol gelatin aqueous slide mounting medium (Sigma). Quantifications were performed blindly using ImageJ software. Briefly, pictures were performed with light microscopy at 400× magnification and were processed using color deconvolution with RGB vectors. The resulting red color images were quantified using a custom threshold (0.173 for RPTEC/hTERT cells and 0.140 for kidney stainings).

### Immunofluorescence (Tissues).

Paraffin-embedded sections (3 μm thick) were deparaffinized with xylene and rehydrated in successive ethanol dilutions. Then, antigen retrieval was done by incubation in sub-boiling 10 mM sodium citrate buffer. Tissues were permeabilized in a 0.4% Triton X-100 solution, and nonspecific binding was blocked with a 5% BSA solution in TBS for 2 hours. Sections were then incubated overnight with an anti-γH2AX antibodies (1:50; no. 9718, Cell Signaling Technology). After washing, the secondary antibody (A10042) was incubated for 45 minutes at room temperature. Again, after washing, the nuclei were stained with a 300 nM DAPI solution (D1306, Life Technologies, Thermo Fisher Scientific). The slides were analyzed using a Zeiss LSM 880 confocal microscope. Quantification was performed using ImageJ. For immunofluorescence studies in free-floating sections, mice were deeply anesthetized with pentobarbital sodium (50 mg/kg, i.p.) and then transcardially perfused with cold NaCl (0.9%) and 4% paraformaldehyde in PBS (pH 7.4). Kidneys were removed, post-fixed for 24 hours in 4% paraformaldehyde, and cryoprotected in 30% sucrose before being frozen at −40° C. in isopentane (methyl-butane) and stored at −80° C. Longitudinal kidney sections (40 μm) were obtained using a Leica cryostat. Free-floating sections were stored in PBSazide (0.2%) at 4° C. Longitudinal kidney sections were incubated with a donkey serum (D9663, MilliporeSigma) at 10% in PBS Triton X-100 (0.2%) for 1 hour and then incubated with anti-A2AR primary antibody (1:50; GP-Af1000, Frontiers Institute) for 72 hours at 4° C. in Signal Boost (8114, Cell Signaling Technology). Alexa Fluor 568-conjugated secondary antibodies (1:500; Life Technologies, Thermo Fisher Scientific) were incubated overnight at room temperature. Lectin staining was performed by incubating sections for 1 hour at room temperature with Lotus tetragonolobus FITC conjugate (Vector Laboratories, FL-1321-2) diluted to 2 μg/mL in blocking medium. Sections were counterstained with DAPI (1:5,000; no. 62247, Thermo Fisher Scientific) and mounted on superfrost slides and left to dry. Then, they were covered with Vectashield Vibrance Antifade Mounting Medium (H-1700, Vector Laboratories). Images were acquired using a Zeiss LSM 710 confocal laser-scanning microscope at ×20 magnification. 3D reconstruction of confocal image stacks was performed using Imaris software (Bitplane).

### Yh2AX Immunostaining.

LLC1 cells were cultured in Lab-tek (15,000 cells/well) and after 24 hours, exposed to cisplatin (2 μM) without or with 10 nM KW-6002 for 6 hours. Cells were fixed with 4% paraformaldehyde, permeabilized with DPBS/0.1% Triton X100 and incubated with γH2AX (Ser139) antibody (1/400; #9718, Cell Signaling Technology) and then, Alexa Fluor 488 secondary antibody (1/200; Life Technologies). Samples were examined on an immunofluorescence microscope (Leica™ DMI8) and γH2AX nuclear foci were counted.

### RNA Extraction.

Total RNA from kidney samples was extracted with phenol/chloroform and subsequently precipitated in isopropanol as described previously (71). Total RNA from cultured renal cells was extracted using RNeasy Mini kit (Qiagen) following manufacturer's recommendations.

### RNA-Sequencing and Analysis.

RNA-seq libraries (n=5-6/group) were generated from 500 ng of total RNA using Illumina® TruSeq® Stranded mRNA Library Prep Kit v2. Briefly, following purification with poly-T oligo attached magnetic beads, the mRNA was fragmented using divalent cations at 94° C. for 2 min. The cleaved RNA fragments were copied into first-strand cDNA using reverse transcriptase and random primers. Strand specificity was achieved by replacing dTTP with dUTP during the second-strand cDNA synthesis by DNA polymerase I and RNase H. Following the addition of a single “A” base and the subsequent ligation of the adapter on double-stranded cDNA fragments, the products were purified and enriched with PCR [30 s at 98° C.; (10 s at 98° C., 30 s at 60° C., 30 s at 72° C.)×12 cycles; 5 min at 72° C.] to create the cDNA library. Surplus PCR primers were further removed by purification using AMPure XP beads (Beckman Coulter), and the final cDNA libraries were checked for quality and quantified using capillary electrophoresis. Sequencing was performed on the Illumina® Genome Hiseq4000 as single-end 50 base reads following Illumina's instructions. Reads were mapped onto the mm 10 assembly of Mus musculus genome using STAR v2.5.3a (72). Only uniquely aligned reads were kept for further analyses. Quantification of gene expression was performed using HTSeq-count v0.6.1p1 (73) and gene annotations from Ensembl releases 90 and 102 and “union” mode. Read counts were normalized across libraries with the method proposed by Ander et al. (74). Comparisons of interest were performed using the test for differential expression proposed by Love et al. (75) and implemented in the DESeq2 Bioconductor library (v1.16.1). Resulting p-values were adjusted for multiple testing using the Benjamini and Hochberg method (76). RNA-sequencing was performed by Plateforme GenomEast, Institut de Génétique et de Biologie Moléculaire et Cellulaire, UMR 7104 CNRS-UdS/INSERM U964 (Illkirch, France). Sequencing data that support the findings of this study have been deposited in GEO with the primary accession code GSE179247.

### Go-Term, STRING and Ingenuity Pathway Analysis.

Functional enrichment analysis was run with DAVID (Database for Annotation, Visualization and Integration Discovery; https://david-d.ncifcrf.gove/home.jsp), STRING Interaction Network (Interaction Networks) or uploaded on the IPA web portal (Qiagen; www.ingenuity.com). The data were analyzed to predict gene networks, molecular and cellular functions, canonical pathways and upstream regulators for cisplatin/KW-modulated genes.

### Gsea Analysis.

GSEA, version 4.1.0 (82, 83), was used, and a preranked analysis was run using the following settings: “No collapsing of gene symbols, the classic enrichment statistic, gene sets containing more than 500 genes and less than 50 genes were excluded from analysis” and with gene sets from Gene Ontology (GO). Genes were ranked on the basis of the values computed as follows: −10×log 10 (P value)×fold change sense.

### Quantitative RT-PCR.

Reverse transcription was performed on 1 μg of RNA using high-capacity cDNA reverse transcription kit (Thermo Fisher), according the manufacturer's recommendation. Real time PCR was performed on a StepOne device using Taqman Gene Expression Master Mix (Thermo Fisher), following manufacturer's recommendations. Expression levels of the following genes were evaluated using comparative CT method (2-deltaCT): Neutrophil Gelatinase Associated Lipocalin (NGAL, Assay IDs Hs00194353_ml and Mm01324470_m1), Kidney Injury Molecule-1 (KIM-1, Assay ID Hs00273334_ml and Mm00506686_m1), Tumor Necrosis Factor alpha (TNFα, Assay ID Hs00174128_m1 and Mm00443258_m1), Interleukin-6 (I16, Assay ID Hs00174131_m1 and Mm00446190_m1), Adora2A Receptor (A_{2A}R, Assay ID Hs00169123_m1 and Mm00802075_m1), Bcl2 associated X (Bax, Assay ID Hs00180269_m1 and Mm00432051_m1), B cell lymphoma 2 (Bcl2, Assay ID Hs00608023_m1 and Mm00477631_m1), Copper transporter receptor 1 (CTR1, Assay ID Hs00741015_m1 and Mm00612987_m1), Organic cation transporter 2 (OCT2, Assay ID Hs00533907 m1 and Mm00457295_m1), ATPase 7A (ATP7A, Assay ID Hs00163707_m1 and Mm00437663_m1), ATPase 7B (ATP7B, Assay ID Hs00163739_m1 and Mm00599675_m1), Multidrug and toxin extrusion ½ (MATE1/2, Assay ID Hs00979028 m1/Hs00945652_m1 and Assay ID Mm00840361_m1/Mm02601002_m1), ATP Binding Cassette Subfamily C Member 2 (Abcc2, Assay ID Hs00166123_m1 and Mm00496899_m1) instructions. Transcript levels of PPIA (human Hs99999904_mland mouse sample Mm02342430_m1) were used as endogenous control.

### Renal Tissue/Cell Concentrations of Platinum.

RPTEC/hTERT cells were cultured in 6-well plates (250,000 cells/well) and exposed to cisplatin (50 μM) without or with KW-6002 (25 μM) for 48 hours. Tissues (35 mg) and cell pellets were first mineralized with hydrochloric acid (30% Suprapur, Merck) or nitric acid (69.5%, Carlo Erba), respectively. Analysis of tissue samples was performed by Graphite Furnace Atomic Absorption Spectrometry (GF-AAS) using an Aanalyst 800 (Perkin Elmer)., while cell samples analysis was performed by Inductively Coupled Plasma Mass Spectrometry (ICP-MS) using an ICAP-Qc (Thermo Scientific). Platinum concentration was finally normalized to the accurately measured kidney mass, or to the number of cells previously assessed.

### Immunoblotting.

Cells and tissues were homogenized using RIPA buffer (Sigma) supplemented with protease and phosphatase inhibitors (Pierce). 10 μg of total proteins were heated for 10 min at 70° C. and loaded on NuPAGE Novex gels (Thermo Fisher Scientific). After transfer of proteins to nitrocellulose membranes, membranes were blocked with 5% milk in TBS-tween followed by incubation with anti-cleaved caspase 3 (1/1000; #9661, Cell Signaling) or anti-cleaved PARP1 (1/1000; #5625, Cell Signaling) primary antibodies. Visualization of protein was achieved by using horseradish peroxidase-coupled secondary antibodies (1/2000; #7074, Cell signaling). Detection signal was performed by using the ECL select chemiluminescent kit (GE Healthcare) and Image Quant LAS 4000 (GE Healthcare). Data were analyzed with Image J. Membranes were probed with anti-GAPDH (glyceraldehyde-3-phosphate deshydrogenase) antibody as normalizer (G9545, Sigma).

### Immunohistochemistry.

For immunohistochemical studies, mice were deeply anesthetized with pentobarbital sodium (50 mg/kg, i.p.), then transcardially perfused with cold NaCl (0.9%) and with 4% paraformaldehyde in PBS (pH 7.4). Kidneys were removed, post-fixed for 24h in 4% paraformaldehyde and cryoprotected in 30% sucrose before being frozen at −40° C. in isopentane (methyl-butane) and stored at −80° C. Longitudinal kidney sections (40 μm) were obtained using a Leica cryostat. Free-floating sections were stored in PBS-azide (0.2%) at 4° C. For immunofluorescent staining, longitudinal kidney sections were incubated with a donkey serum (Sigma-Aldrich, D9663) at 10% in PBS Triton X100 (0.2%) for 1 h and then incubated with anti-A_{2A} receptor primary antibody (1/200; GP-Af1000, Frontiers Institute) overnight at 4° C. Alexa Fluor 568-conjugated secondary antibody (1/500, Life Technologies) was incubated for one hour at room temperature. Sections were counterstained with DAPI (1/5000, 62247, Thermoscientific) and were mounted on superfrost slides and leaved to dry. Then, they were incubated with TrueView Autofluorescence Quenching kit (Vector Laboratories, SP-8400) for 5 min at room temperature to remove autofluorescence and were covered with Vectashield Vibrance Antifade Mounting Medium (Vector Laboratories, H-1700). Images were acquired using a Zeiss LSM 710 confocal laser-scanning microscope at 20× magnification. 3D reconstruction of confocal image stacks was performed using Imaris software (Bitplane).

### Statistics.

All data are presented as mean±SEM. Differences between groups were assessed using two-tailed Student's t-test, One-Way ANOVA followed by multiple comparison Tukey's post-hoc test or repeated measures two-way ANOVA using Prism (GraphPad software). Differences were considered statistically significant at p<0.05. The number of biologically independent experiments, sample size, p values, and statistical tests are all indicated in the main text or figure legends.

### Results

Cisplatin-Induced Nephrotoxicity is Associated with Renal AZAR Upregulation in Mice.

Mice treated with cisplatin either acutely (Acute “A” model; a single dose of 10 mg/kg; FIG. 1A or sub-chronically (Sub-chronic “SC” model; 3 mg/kg for six days; FIG. 1B), exhibited marked renal dysfunction, as shown by increased blood urea nitrogen (BUN) levels (data not shown) as well as severe histological lesions (data not shown), including the presence of necrotic cells and tubular casts. Accordingly, mRNA levels of two renal injury markers, NGAL (neutrophil Gelatinase Associated Lipocalin) and KIM-1 (Kidney Injury Molecule 1), were significantly increased (data not shown). Cisplatin nephrotoxicity was associated with an inflammatory response and apoptosis, as exemplified by the increased mRNA expression of Il6 and Tnf (data not shown), and the enhanced Bax/Bcl-2 ratio (data not shown), as previously described (35).

Interestingly, we observed that cisplatin promoted the upregulation of A_{2A}R (data not shown). Immunohistofluorescence showed that under cisplatin, A_{2A}R is present in renal resident cells and especially in proximal epithelial tubular cells (data not shown). Furthermore, A_{2A}R levels were significantly correlated to BUN (r2=0.63, p<0.0001) as well as to NGAL (r2=0.71, p<0.0001) and KIM-1 (r2=0.69, p<0.0001) expression (data not shown). These data suggest that A_{2A}R dysregulation might be associated with the pathological processes underlying cisplatin-induced nephrotoxicity.

### AZAR Antagonism Alleviates Cisplatin-Induced Toxicity In Vivo and In Vitro.

To assess whether A_{2A}R function is indeed involved in cisplatin-induced renal injury, we evaluated the impact of the FDA-approved selective A_{2A}R antagonist istradefylline (KW-6002) in both the acute (“A”) and sub-chronic (“SC”) models of cisplatin-induced kidney toxicity (FIGS. 1C and 1D). KW-6002 significantly mitigated renal dysfunction induced by cisplatin, as shown by the significant reduction of BUN (A model: −42.9±7.4%; SC model: −70.2±5.1% vs. cisplatin; FIGS. 2A, 2G and 5), NGAL (A: −55.5±5.9%; SC: −82.9±1.4% vs. cisplatin; FIGS. 2C, 2I) and KIM-1 (A: −95.2±0.7%; SC: −79.5±3.8% vs. cisplatin; FIGS. 2D, 2J) expression as well as less severe histological lesions (FIGS. 2B, 2H). KW-6002 treatment also significantly reduced renal inflammation as shown by the lower mRNA levels of Tnfα (A: −36.0±4.3%; FIGS. 2E, 2K) and I16 (A: −73.8±5.3%; SC: −77.5±5.6% vs. cisplatin; FIGS. 2F, 2L).

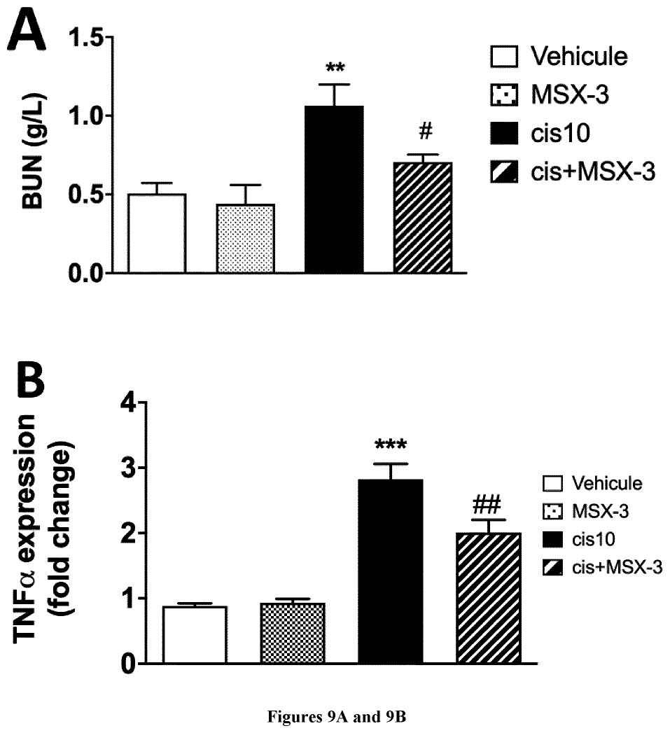

MSX-3 significantly mitigated renal dysfunction induced by cisplatin, as shown by the significant reduction of BUN (FIG. 9A). MSX-3 treatment also significantly reduced renal inflammation as shown by the lower mRNA levels of Tnfα (FIG. 9B).

In line with the association of lipid accumulation and lipotoxicity with kidney diseases and consistent with recent works (36-37), we also observed that cisplatin induced an accumulation of lipid droplets that was significantly reduced by KW-6002 (data not shown).

We next used a human proximal tubular epithelial cell line (RPTEC/hTERT1) to detail the effect of KW-6002 against cisplatin renal toxicity, in vitro. In agreement with our in vivo data, KW-6002 reduced cisplatin-induced cell death in a concentration-dependent manner (data not shown). KW-6002 particularly inhibited cisplatin-induced apoptosis (data not shown). In addition, lipid accumulation induced by cisplatin in RPTEC/hTERT1 cells was also significantly reduced by KW-6002 (data not shown). Taken together, these in vivo and in vitro data strongly support the ability of KW-6002 to protect from cisplatin-induced kidney injury.

Transcriptomic Signature Associated with the Protective Effect of KW-6002 on Cisplatin-Induced Renal Injury.

To gain insights into the in vivo molecular events underlying the beneficial effects of A_{2A}R antagonism in the injured kidney, we used a RNA-Seq transcriptomic approach in the sub-chronic protocol. Principal Component Analysis (PCA) of the experimental groups (n=5-6 per group).

Cisplatin profoundly affected the kidney transcriptome, impacting the expression of 4649 genes (adjusted p-value<0.01, log 2 fold change±1), 2350 being upregulated and 2299 downregulated (data not shown). Interestingly, KW-6002 reduced by ˜50% the transcriptomic changes induced by cisplatin (data not shown). Among the 2350 genes upregulated by cisplatin, 811 (˜34%) were normalized by KW-6002 co-administration (data not shown), while KW-6002 had almost no effect on control mice (data not shown). Functional enrichment analyses done using DAVID (https://david.ncifcrf.gov) showed that these 811 genes were associated with cell adhesion and proliferation (data not shown). Likewise, among the 2299 genes downregulated by cisplatin, 635 (˜27%) were normalized by KW-6002 co-treatment (data not shown), which were mainly associated with oxidation-reduction reactions and transport processes (data not shown). We then used Ingenuity Pathway Analysis (IPA) to predict the most significant molecular and cellular functions, toxicological features or upstream regulators affected by cisplatin that were normalized by KW-6002 co-treatment. Our analysis of these 635 KW-6002-modulated genes identified networks and canonical pathways involved in metabolism of amino acids, molecular transport (data not shown), kidney injury and lipid metabolism (including fatty acid metabolism, data not shown), suggesting that the genes in these networks were specifically associated with the protective effects of KW-6002 in cisplatin-treated kidneys. Next, we performed the upstream analysis of KW-6002-modulated genes and identified several crucial upstream regulators that are known to affect kidney functions and/or lipid homeostasis (data not shown). Specifically, our analysis predicted activation of Hepatocyte Nuclear Factor 1 A-isoform (HNF1A, data not shown). Our analysis further predicted the activation of Lim Homeobox protein 1 (LHX1, data not shown) or Synuclein (SNCA, data not shown).

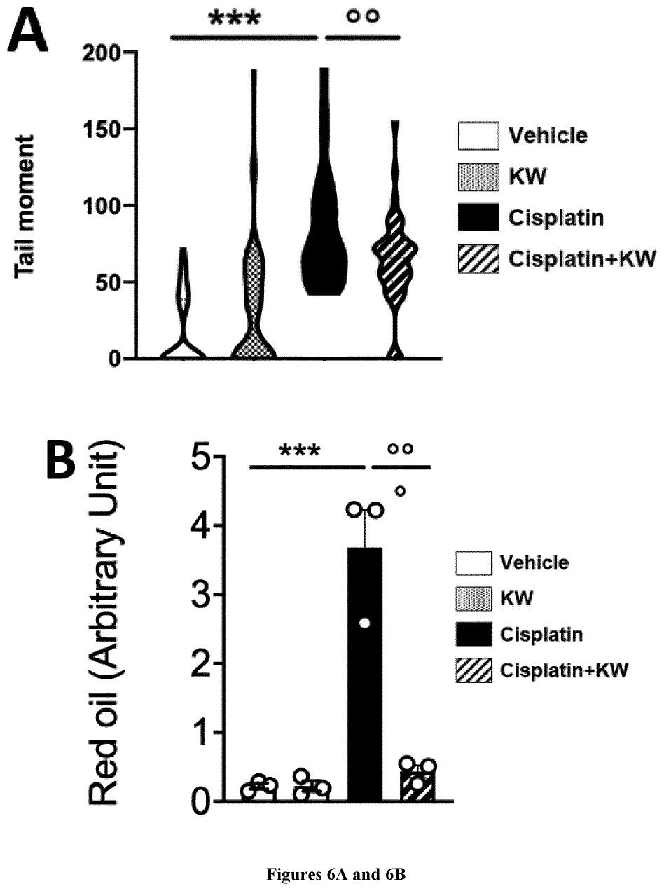

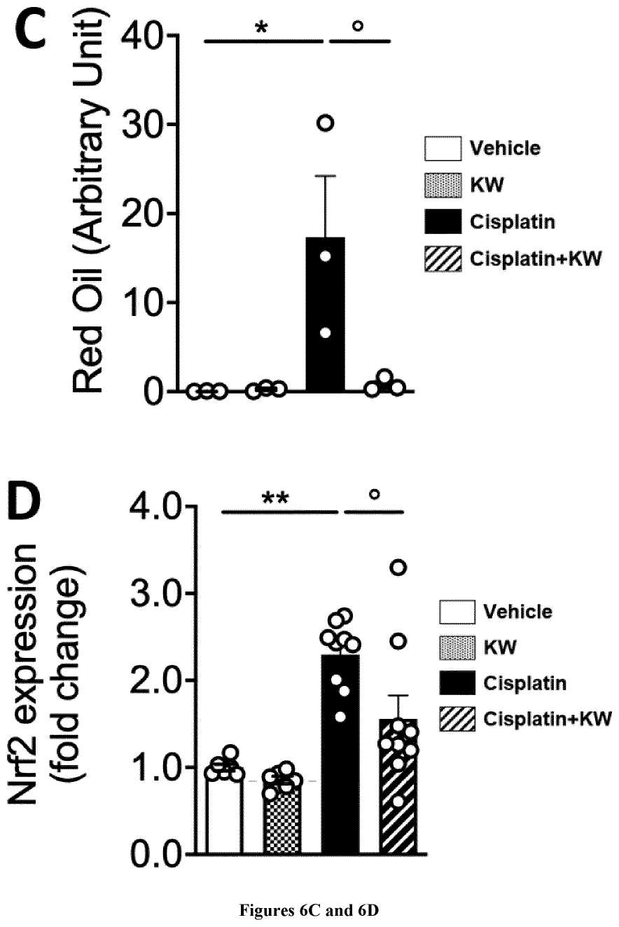

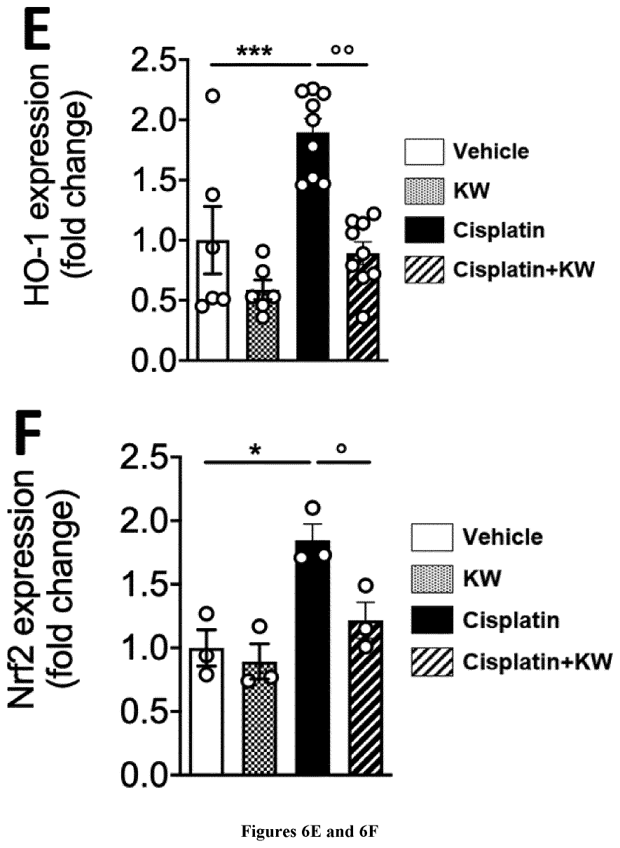

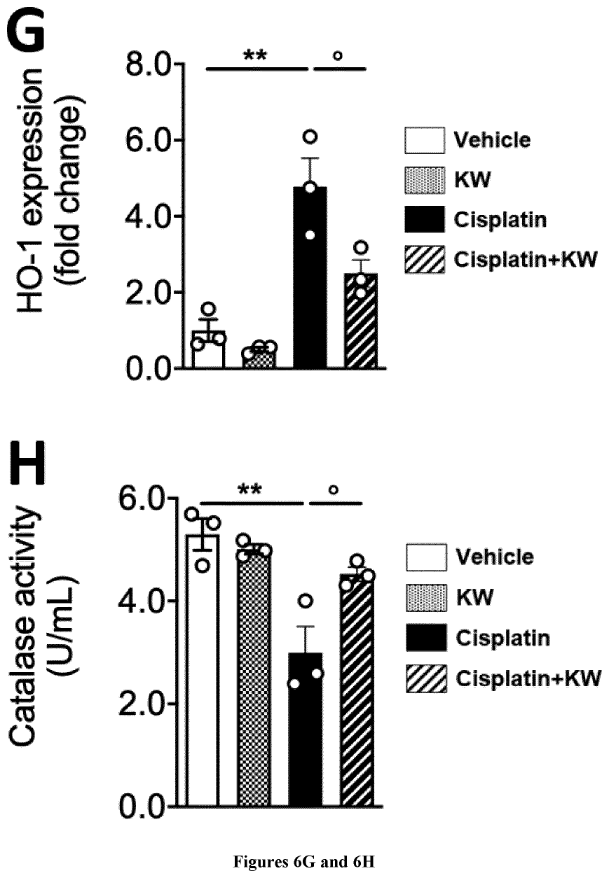

The above-mentioned KW6002-regulated pathways uncovered by RNA-Seq analysis were confirmed by additional in vitro and in vivo analyses. First, using a human proximal tubular epithelial cell line (RPTEC/hTERT1), we found that KW6002 concentration-dependently reduced cisplatin-induced cell death (data not shown), in particular by inhibiting cisplatin-induced apoptosis (data not shown) as well as DNA damage (FIG. 6A). Furthermore, cisplatin-induced lipid accumulation was significantly reduced by KW6002 in both RPTEC/hTERT1 cells (FIG. 6B) and kidney samples (FIG. 6C). Finally, KW6002 normalized the cisplatin-induced increase in the expression of 2 master regulators of oxidative stress—Nrf2 and HO1—both in vivo (FIGS. 6D and 6E) and in vitro (FIGS. 6F and 6G) as well as the decrease in catalase activity (FIG. 6H).

Overall, these are important observations supporting the connection between KW-6002 effects and key protective signaling events in cisplatin-treated kidneys.

### Effect of KW-6002 on the Antitumoral Properties of Cisplatin.

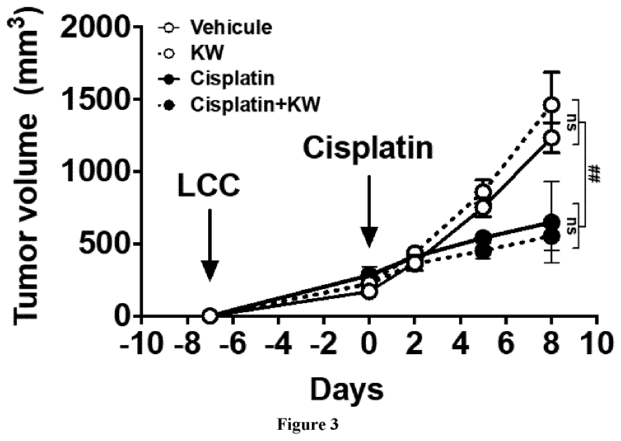

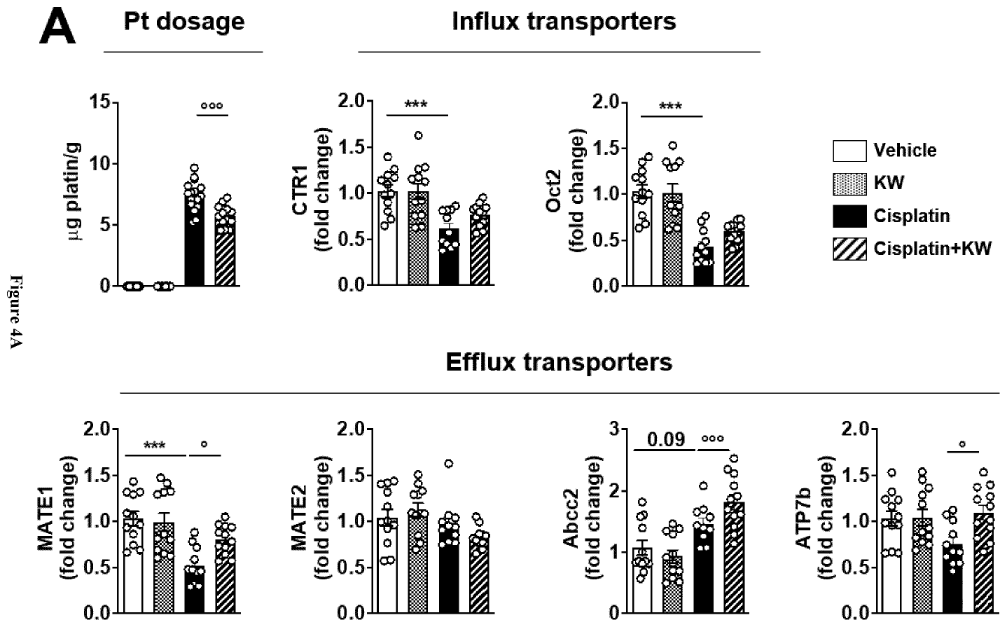

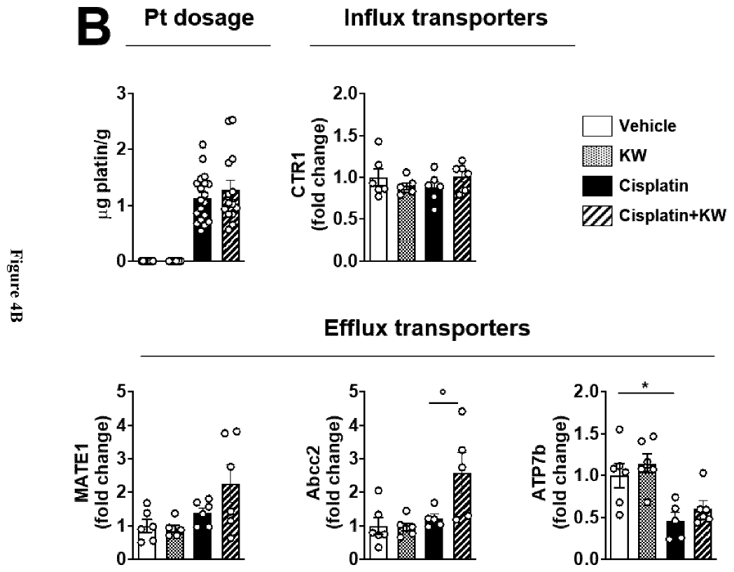

From a clinical perspective, it is crucial to determine whether KW-6002 preserved kidney function after cisplatin exposure, without, at minima, compromising its anti-tumoral efficacy. To address this question, we used the syngeneic LLC1 lung cancer mouse model. Subcutaneous LLC1 tumors were induced in C57BL6/J mice, which were then treated with cisplatin, KW-6002 or both (FIG. 1E). In this model, we could then simultaneously gauge if KW-6002 modulated the effects of cisplatin towards kidney injury and tumorigenicity. We observed that the ability of KW-6002 to protect from cisplatin-induced kidney injury was preserved in tumor-bearing mice (data not shown). Importantly, KW-6002 did not compromise the anti-tumoral response to cisplatin (FIG. 3). Moreover, a whole transcriptome analysis was conducted to decipher potential molecular changes occurring in tumors in response to cisplatin and/or KW-6002. PCA (n=5 per group) (data not shown). Compared with vehicle-treated mice, KW-6002 did not modulate gene expression in tumors (not shown). Compared with vehicle-treated mice, co-treatment of mice with cisplatin and KW-6002 changed the expression of 3923 genes (adjusted p-value<0.05, log 2 fold change±0.32), while cisplatin alone altered the transcription of 1801 genes. Therefore, the impact of cisplatin on tumor cell transcriptome was enhanced in the presence of KW-6002 (data not shown). Among the 2497 (out 3923) genes selectively modulated by cisplatin/KW-6002 co-treatment vs. vehicle, 1016 genes were downregulated whose annotation particularly referred to chemokine/cytokine responses, cell cycle as well as DNA repair and replication (data not shown). Using IPA, we further determined the most significant diseases, molecular and cellular functions as well as biological networks related to these 1016 genes that are specifically downregulated by KW-6002 upon cisplatin co-treatment. The altered gene expression patterns were related to disorders including cancer and immunological disease (data not shown). In addition, the cellular and molecular functions affected by KW-6002 were predicted to be involved in “cell death and survival, cellular development, cellular growth and proliferation”, including increased necrosis (p=9.55×10−24) and apoptosis (p=2.78×10−22). Furthermore, our analysis of the genes that are specifically downregulated by KW-6002 upon cisplatin co-treatment identified 25 biological gene networks (data not shown), the 5 most significant being related to “Protein Synthesis, RNA Damage and Repair, RNA Post-Transcriptional Modification”, “Cancer, Hematological Disease, Immunological Disease” and “Antimicrobial Response, Infectious Diseases, Inflammatory Response”, “Cancer, Cardiovascular Disease, DNA Replication, Recombination, and Repair” and “Molecular Transport, Post-Translational Modification, RNA Trafficking” (data not shown). These transcriptomic data are suggestive of a potential molecular synergistic anti-tumoral and/or anti-proliferative effect of KW-6002 when administrated in combination with cisplatin. In accordance with these transcriptomic analyses, our in vitro data suggest that KW-6002 likely increases LLC1 sensitivity to cisplatin, including apoptosis and/or DNA damage (data not shown).

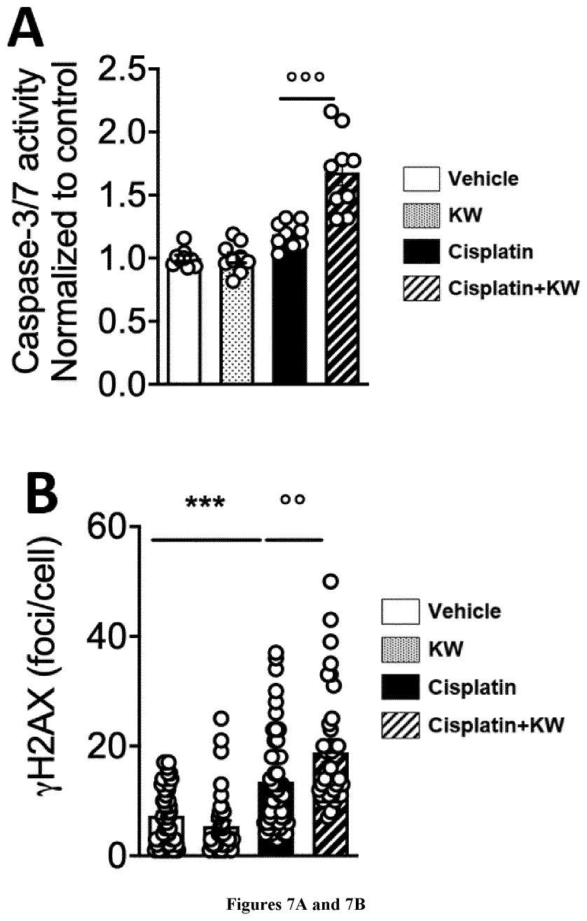

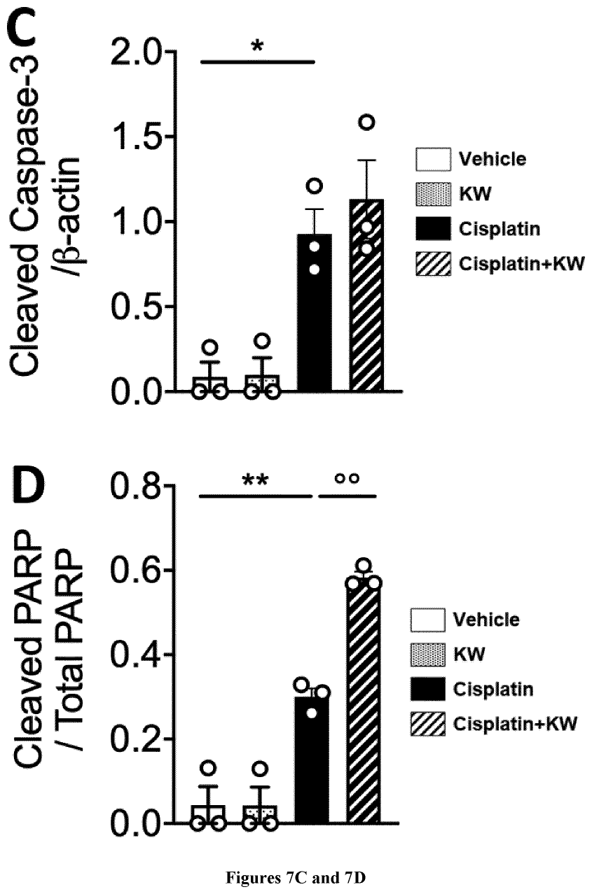

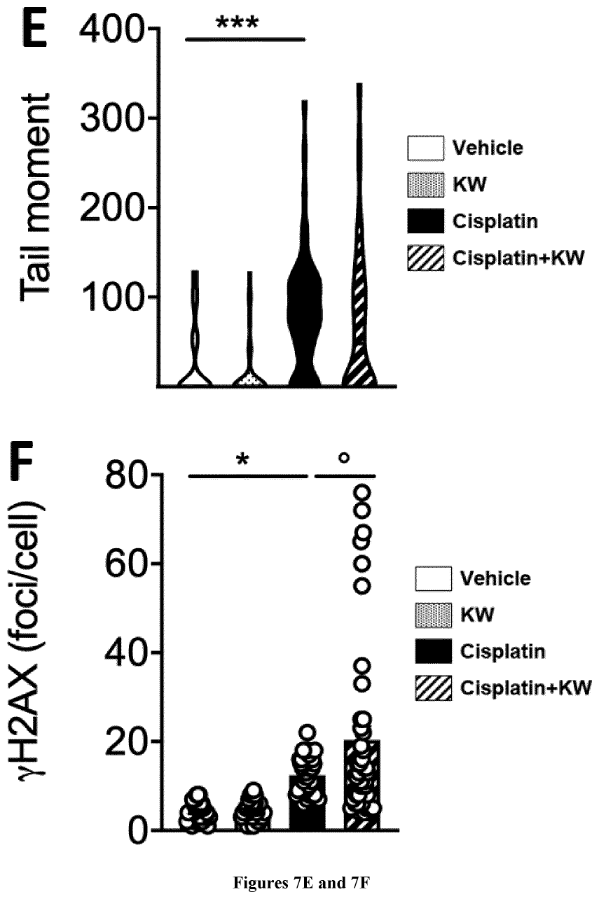

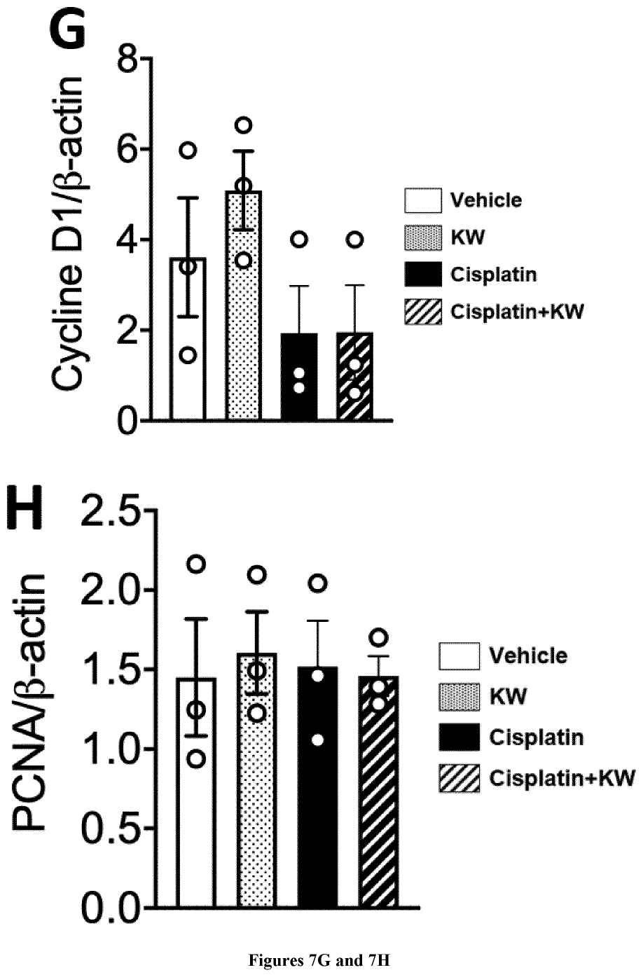

In agreement with this pathway analysis, in vitro studies performed using 2 cancerous cell lines (LLC1 and H1975) confirmed that KW6002 did not impede the antitumoral effect of cisplatin in terms of apoptosis (FIGS. 7A, 7C and 7D), DNA damage (FIGS. 7B, 7E, and 7F), and the cell cycle (FIGS. 7G and 7H). Of note, KW6002 even potentiated the efficacy of cisplatin (FIGS. 7A-7B, 7D, 7F, 7H and 7I), with a particular effect on cisplatin-induced DNA damage, as evidenced by the increased number of H2AX+ cells observed both in vitro in LLC1 and H1975 cells (FIGS. 7B and 7F) as well as in vivo in the LLC1 syngeneic model (FIG. 71).

### KW-6002 Reduced Renal Accumulation of Cisplatin.

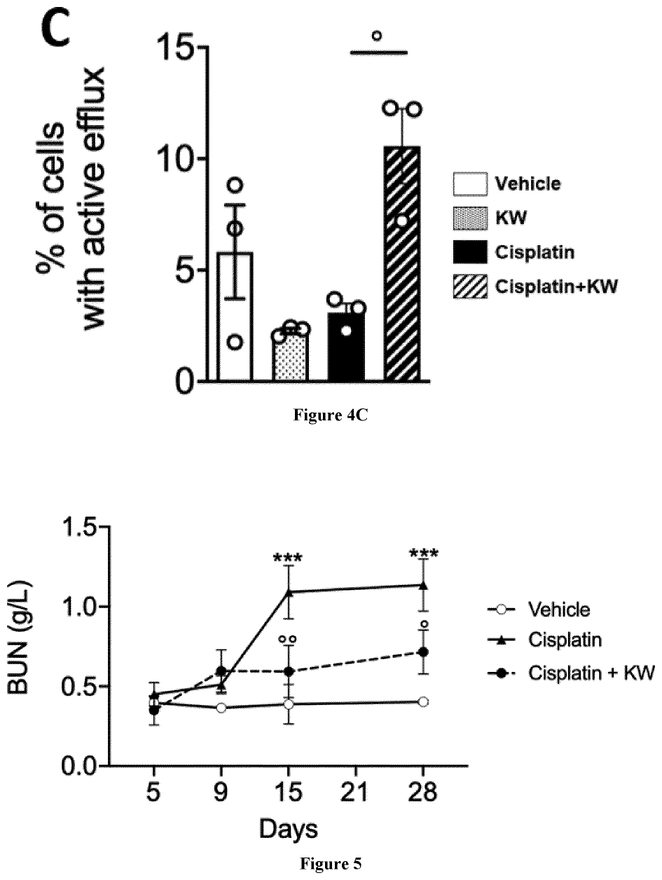

To understand how KW-6002 alleviates cisplatin-induced nephrotoxicity while preserving the ability of cisplatin to control tumor growth, platinum levels were quantified in both kidney and tumors. This analysis revealed that in KW-6002-treated mice, cisplatin accumulation was lowered in the kidney while it remained unchanged in the tumors (FIGS. 4A and 4B). The effect of A_{2A}R antagonism on platinum accumulation in kidneys was replicated in an independent experiment (data not shown; from animals described on FIGS. 2G-2L). The differential effect of KW-6002 on the accumulation of cisplatin in kidneys and tumors led us to evaluate, in both tissues, the mRNA levels of several transporters known to be involved in cisplatin uptake (CTR1 and OCT2) and efflux (ATP7B, MATE1/SLC47A1, MATE2 and ABCC2) (6). In kidneys, using qPCR analysis (FIG. 4A) but also data from our RNA-Seq experiments (data not shown), we observed that the expression of some efflux transporters was significantly increased by KW-6002. By contrast, no expression changes were found in tumors (FIG. 4B).

Moreover, this observed discrepancy between renal and cancer cells led us to evaluate the efflux ability of renal RPTEC/hTERT1 and cancer H1975 cells by flow cytometry. While efflux was significantly reduced by KW6002 in cancer cells in response to cisplatin (data not shown), it was strongly enhanced by KW6002 in cisplatin-treated RPTEC/hTERT1 cells (FIG. 4C).

A2AR Antagonism Protects Against Cisplatin-Induced Nephrotoxicity and CIPN (Chemotherapy-Induced Peripheral Neuropathy) while Enhancing Tumor Growth Control in a Syngeneic Model of HPV+ Squamous Carcinoma.

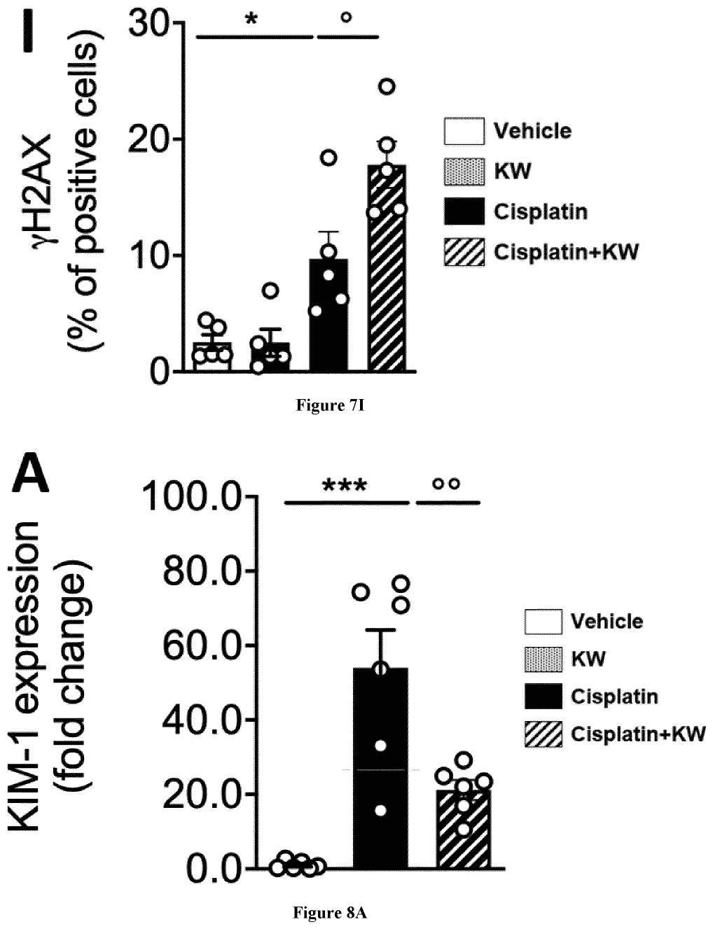

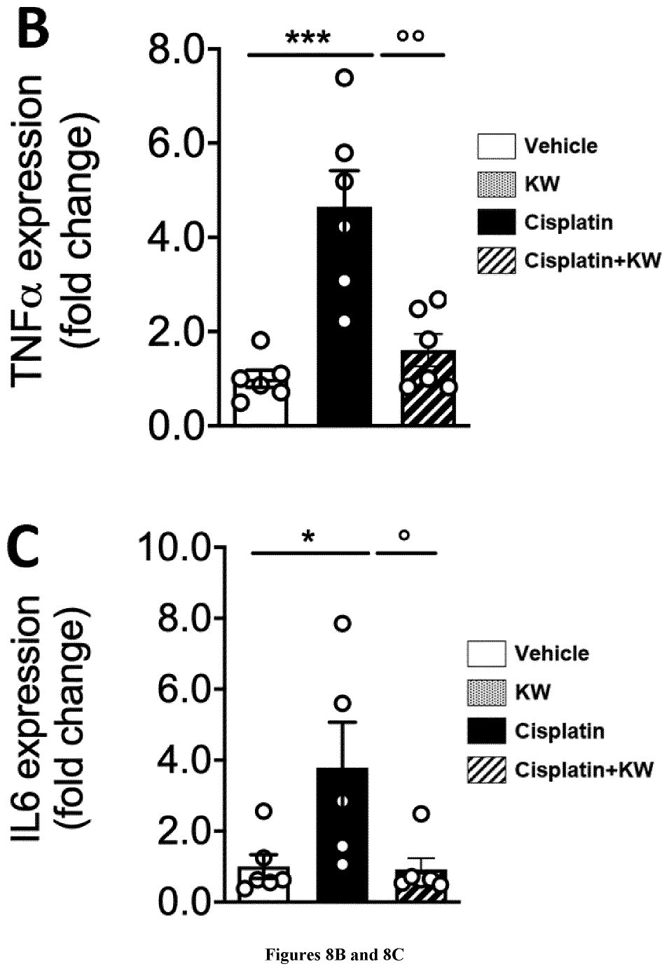

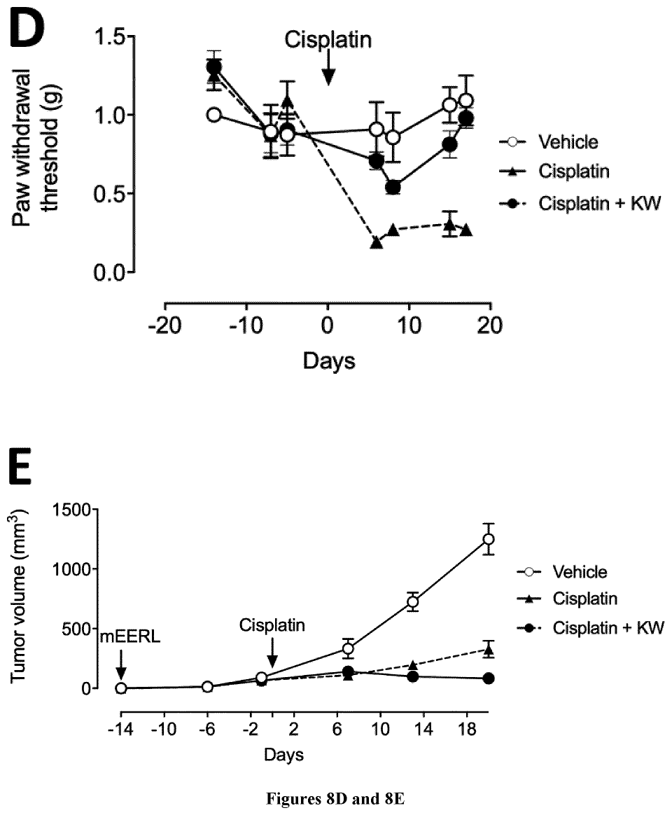

We validated the nephro- and neuro protective effects of KW6002 in a tumoral context using an additional cancer mouse model (77). Subcutaneous mEERL cells were injected into C57B16/J mice, which were then treated with cisplatin alone or in combination with KW6002 (data not shown). KW6002 administration in tumor-bearing mice limited indicators of renal toxicity (KIM-1, FIG. 8A) and expression of the inflammatory cytokines Tnf and I16 (FIGS. 8B and 8C) induced by cisplatin. KW6002 also alleviated cisplatin-induced pain hypersensitivity in this model (FIG. 8D). Finally, KW6002 significantly potentiated tumor control by cisplatin (FIG. 8E). Overall, using this additional model with a different cancer etiology, the nephroprotective effect, the reduction of pain hypersensitivity, and the potentiation of tumor control were replicated, highlighting the promising therapeutic potential of A_{2A}R inhibition.

### DISCUSSION

Cisplatin-induced nephrotoxicity remains serious adverse effects, affecting approximately one-third of exposed patients (11-12). Identifying targets to alleviate such toxicities without lessening tumor control by cisplatin is therefore a major clinical challenge. Moreover, an optimal therapeutic solution would ideally act synergistically with cisplatin to promote cancer regression while protecting kidney and sensory functions. In the present study, we provide evidence that administration of the FDA-approved A_{2A}R antagonist istradefylline (KW-6002) efficiently and reproducibly prevents cisplatin-induced nephrotoxicity in mice. These beneficial effects were observed while the tumor-growth control properties of cisplatin were preserved, with even an enhancement of some anti-tumoral molecular pathways.

Our targeted and non-targeted (RNA-Seq) experiments indicated that cisplatin profoundly affects renal function by promoting cell death via multiple pathways including inflammatory response, oxidation and reduction reactions, intracellular lipid accumulation, transport impairment and apoptosis induction (40-42). Treatment with KW-6002 alleviated most of them. Whether the mechanisms underlying KW-6002 actions in the DRG are similar to those in the kidney will be the focus of future studies.

Antagonists of A_{2A}R have previously been shown to mitigate injury by decreasing oxidative stress in different cells types and tissues (43-47). The effect of KW-6002 on renal oxidation-reduction mechanisms is of particularly importance since, once in the cytosol, cisplatin is highly reactive towards nucleophilic substances such as glutathione (GSH), cysteines or methionines, which are metabolically activated to form reactive thiols (2, 48). Accumulation of cisplatin in the mitochondria of tubular epithelial cells then increases the levels of reactive oxygen species and decreases the levels of anti-oxidant components such as GSH and superoxide dismutase (40, 49), leading to oxidative stress-related damages and death of proximal tubular epithelial cells (50-51).

Our data also suggest that the nephroprotective effect of KW-6002 relies on its ability to limit platinum accumulation in the kidney. The latter is consistently associated with the upregulation of ABCC2, ATP7b and MATE-1 (Multidrug and toxin extrusion 1), the main transporters previously reported to be involved in cisplatin efflux (6). In line with this, upregulation of MATE-1 has been shown to increase the efflux of cisplatin from renal cells (52), while genetic deletion of MATE-1 exacerbates cisplatin nephrotoxicity in mice (53). How KW-6002 regulates efflux transporters expression remains unclear; however, A_{2A}R activation was previously reported to decrease the expression and function of P-glycoprotein (ABCB1), a member of the same family than ABCC2, leading to the accumulation of P-glycoprotein substrates in the mouse brain (54). The localization of A_{2A}R in renal resident cells and especially in epithelial tubular cells of cisplatin-treated animals and the fact that KW-6002 limits the accumulation of cisplatin in RPTEC/hTERT1 proximal tubular epithelial cell line in vitro (data not shown) speak for a direct effect of KW-6002 on A_{2A}R expression on tubular cells. However, we cannot rule out that KW-6002 exerts its beneficial effect by modulating A_{2A}R receptors located on inflammatory cells, as supported by the reduced levels of Tnf and Il-6 expression. Extracellular adenosine has been indeed shown to be important for the regulation of immune cell activation in the kidney, in particular in the context of renal ischemia; however, activation rather than blockade of A_{2A}R signaling is acknowledged for its immunosuppressive effect (30-31, 55).

It is clinically highly relevant that KW-6002 exerts potent effects towards cisplatin-induced renal toxicity without affecting its anti-tumoral properties. Indeed, reduced tumor growth rate induced by cisplatin was not affected by KW-6002 co-treatment. Adenosine levels are particularly elevated in the tumor micro-environment (56-57Ohta et al., 2006; 57), impairing antitumor immunity, notably through the activation of A_{2A}R expressed by immune cells (13-58). Accordingly, A_{2A}R antagonists are currently being explored in clinical trials as co-adjuvants for auto-immune transplant therapies for immunogenic cancers (https://www.cancer.gov/about-cancer/treatment/clinical-trials/intervention/adenosine-a2a-receptor-antagonist-cpi-444). Interestingly, platinum-based chemotherapeutic agents have been suggested to promote adenosine surge by cancer cells, conferring chemoresistance and further suppressing anti-tumor immunity (58). In this context, A_{2A} receptor blockade is currently seen as a valuable strategy to improve chemotherapy and exert immune-oncological effects (58-59). Remarkably, the molecular analysis of syngeneic tumors from animals treated with cisplatin demonstrated that co-treatment with KW-6002 led to a major reducing impact on molecular pathways related to cancer. Our RNA sequencing data also highlighted that, in the context of cisplatin co-treatment, KW-6002 also altered patterns of genes involved with carcinogenesis, notably related to cell growth, such as DNA replication and cell cycle. Interestingly, IPA from in vivo tumors also predicted necrosis and apoptosis to be particularly activated in tumors (P=2.78×10−22), in agreement with our in vitro experiments supporting a synergic effect of cisplatin and KW-6002 on LLC1 cells. Taken together, our data suggested that KW-6002 bolsters the anti-tumoral properties of cisplatin through A_{2A}R-mediated control of tumor-immune-inflammatory interactions. These observations are in agreement with previous data highlighting that caffeine, a non-selective adenosine receptor antagonist, potentiate the anti-tumoral effect of cisplatin both in vitro and in vivo (60-61).

In conclusion, our study clearly demonstrates the high efficiency of KW-6002 to attenuate cisplatin-induced nephrotoxicity without compromising its anti-tumoral properties. Considering the safety and tolerability of the FDA-approved KW-6002, demonstrated in previous clinical trials targeting Parkinson's disease patients (62), our data prompt its clinical repurposing in patients with cancer undergoing cisplatin chemotherapy.

### REFERENCES

Throughout this application, various references describe the state of the art to which this invention pertains. The disclosures of these references are hereby incorporated by reference into the present disclosure.
1. Dasari S, Tchounwou P B. Cisplatin in cancer therapy: molecular mechanisms of action. Eur J Pharmacol. 2014; 740:364-378.
2. Wang D, Lippard S J. Cellular processing of platinum anticancer drugs. Nat Rev Drug Discov. 2005; 4 (4): 307-320.
3. Miller R P, et al. Mechanisms of Cisplatin nephrotoxicity. Toxins (Basel). 2010; 2 (11): 2490-2518.
4. Rybak L P, Whitworth C A. Ototoxicity: therapeutic opportunities. Drug Discov Today. 2005; 10 (19): 1313-1321.
5. Staff N P, et al. Chemotherapy-induced peripheral neuropathy: A current review. Ann Neurol. 2017; 81 (6): 772-781.
6. Ciarimboli G. Membrane transporters as mediators of Cisplatin effects and side effects. Scientifica (Cairo). 2012; 2012:473829.
7. Manohar S, Leung N. Cisplatin nephrotoxicity: a review of the literature. J Nephrol. 2018; 31 (1): 15-25.
8. Oh G S, et al. Cisplatin-induced Kidney Dysfunction and Perspectives on Improving Treatment Strategies. Electrolyte Blood Press. 2014; 12 (2): 55-65.
9. Pabla N, Dong Z. Cisplatin nephrotoxicity: mechanisms and renoprotective strategies. Kidney Int. 2008; 73 (9): 994-1007.
10. Liu H E, et al. Multiple analytical approaches demonstrate a complex relationship of genetic and nongenetic factors with cisplatin- and carboplatin-induced nephrotoxicity in lung cancer patients. Biomed Res Int. 2014; 2014:937429.
11. Hamroun A, et al. Prevention of cisplatin-induced acute kidney injury: a systematic review and meta-analysis. Drugs. 2019; 79 (14): 1567-1582.
12. Cavaletti G, Marmiroli P. Chemotherapy-induced peripheral neurotoxicity. Nat Rev Neurol. 2010; 6 (12): 657-666.
13. Antonioli L, et al. Immunity, inflammation and cancer: a leading role for adenosine. Nat Rev Cancer. 2013; 13 (12): 842-857.
14. Chen J F, Eltzschig H K, Fredholm B B. Adenosine receptors as drug targets-what are the challenges?. Nat Rev Drug Discov. 2013; 12 (4): 265-286.
15. Haskó G, et al. Adenosine receptors: therapeutic aspects for inflammatory and immune diseases. Nat Rev Drug Discov. 2008; 7 (9): 759-770.
16. Jacobson K A, Gao Z G. Adenosine receptors as therapeutic targets. Nat Rev Drug Discov. 2006; 5 (3): 247-264.
17. Jacobson K A, Gao Z G, Matricon P, Eddy M T, Carlsson J. Adenosine A_{2A} receptor antagonists: from caffeine to selective non-xanthines. Br J Pharmacol. 2020; 10.1111/bph.15103. doi: 10.1111/bph.15103
18. Cunha R A. How does adenosine control neuronal dysfunction and neurodegeneration?. J Neurochem. 2016; 139 (6): 1019-1055.
19. Xiao C, Liu N, Jacobson K A, Gavrilova O, Reitman M L. Physiology and effects of nucleosides in mice lacking all four adenosine receptors. PLOS Biol. 2019; 17 (3): e3000161.
20. Oyarzún C, Garrido W, Alarcón S, et al. Adenosine contribution to normal renal physiology and chronic kidney disease. Mol Aspects Med. 2017; 55:75-89.
21. Dwyer K M, Kishore B K, Robson S C. Conversion of extracellular ATP into adenosine: a master switch in renal health and disease. Nat Rev Nephrol. 2020; 16 (9): 509-524.
22. Lee H T, et al. A1 adenosine receptor activation inhibits inflammation, necrosis, and apoptosis after renal ischemia-reperfusion injury in mice. J Am Soc Nephrol. 2004; 15 (1): 102-111.
23. Grenz A, et al. The reno-vascular A2B adenosine receptor protects the kidney from ischemia. PLOS Med. 2008; 5 (6): e137.
24. Park S W, et al. Proximal tubule sphingosine kinase-1 has a critical role in A1 adenosine receptor-mediated renal protection from ischemia. Kidney Int. 2012; 82 (8): 878-891.
25. Roberts V S, et al. The role of adenosine receptors A2A and A2B signaling in renal fibrosis. Kidney Int. 2014; 86 (4): 685-692.
26. Sheth S, et al. Adenosine receptors: expression, function and regulation. Int J Mol Sci. 2014; 15 (2): 2024-2052.
27. Gill A, et al. Protective effect of tonapofylline (BG9928), an adenosine A1 receptor antagonist, against cisplatin-induced acute kidney injury in rats. Am J Nephrol. 2009; 30 (6): 521-526.
28. Knight R J, Collis M G, Yates M S, Bowmer C J. Amelioration of cisplatin-induced acute renal failure with 8-cyclopentyl-1,3-dipropylxanthine. Br J Pharmacol. 1991; 104 (4): 1062-1068.
29. Nagashima K, Kusaka H, Karasawa A. Protective effects of KW-3902, an adenosine A1-receptor antagonist, against cisplatin-induced acute renal failure in rats. Jpn J Pharmacol. 1995 April; 67 (4): 349-57.
30. Day Y J, et al. Renal protection from ischemia mediated by A2A adenosine receptors on bone marrow-derived cells. J Clin Invest. 2003; 112 (6): 883-891.
31. Vincent I S, Okusa M D. Adenosine 2A receptors in acute kidney injury. Acta Physiol (Oxf). 2015; 214 (3): 303-310.
32. Garcia G E, et al. Adenosine A2A receptor activation prevents progressive kidney fibrosis in a model of immune-associated chronic inflammation. Kidney Int. 2011; 80 (4): 378-388.
33. Awad A S, et al. Adenosine A2A receptor activation attenuates inflammation and injury in diabetic nephropathy. Am J Physiol Renal Physiol. 2006; 290 (4): F828-F837.
34. Garcia G E, et al. Adenosine A2A receptor activation and macrophage-mediated experimental glomerulonephritis. FASEB J. 2008; 22 (2): 445-454.
35. Holditch S J, et al. Recent advances in models, mechanisms, biomarkers, and interventions in cisplatin-induced acute kidney injury. Int J Mol Sci. 2019; 20 (12): 3011.
36. Oh C J, et al. Pyruvate dehydrogenase kinase 4 deficiency attenuates cisplatin-induced acute kidney injury. Kidney Int. 2017; 91 (4): 880-895.
37. Li S, et al. Reduced kidney lipoprotein lipase and renal tubule triglyceride accumulation in cisplatin-mediated acute kidney injury. Am J Physiol Renal Physiol. 2012; 303 (3): F437-F448.
38. Sisignano M, et al. Mechanism-based treatment for chemotherapy-induced peripheral neuropathic pain. Nat Rev Neurol. 2014; 10 (12): 694-707.
39. Krukowski K, et al. HDAC6 inhibition effectively reverses chemotherapy-induced peripheral neuropathy. Pain. 2017; 158 (6): 1126-1137.
40. Santos N A, et al. Cisplatin-induced nephrotoxicity is associated with oxidative stress, redox state unbalance, impairment of energetic metabolism and apoptosis in rat kidney mitochondria. Arch Toxicol. 2007; 81 (7): 495-504.
41. Freitas-Lima L C, et al. PPAR-a deletion attenuates cisplatin nephrotoxicity by modulating renal organic transporters MATE-1 and OCT-2. Int J Mol Sci. 2020; 21 (19): 7416.
42. Cohen A, et al. Mechanism and reversal of drug-induced nephrotoxicity on a chip. Sci Transl Med. 2021; 13 (582): eabd6299.
43. Aires I D, et al. Blockade of microglial adenosine A2A receptor suppresses elevated pressure-induced inflammation, oxidative stress, and cell death in retinal cells. Glia. 2019; 67 (5): 896-914.
44. Bortolatto C F, et al. Selective A2A receptor antagonist SCH 58261 modulates striatal oxidative stress and alleviates toxicity induced by 3-Nitropropionic acid in male Wistar rats. Metab Brain Dis. 2017; 32 (6): 1919-1927.
45. Leite M R, Wilhelm E A, Jesse C R, Brandão R, Nogueira C W. Protective effect of caffeine and a selective A2A receptor antagonist on impairment of memory and oxidative stress of aged rats. Exp Gerontol. 2011; 46 (4): 309-315.
46. Nobre H V Jr, et al. Caffeine and CSC, adenosine A2A antagonists, offer neuroprotection against 6-OHDA-induced neurotoxicity in rat mesencephalic cells. Neurochem Int. 2010; 56 (1): 51-58.
47. Thakur S, Du J, Hourani S, Ledent C, Li J M. Inactivation of adenosine A2A receptor attenuates basal and angiotensin I I-induced ROS production by Nox2 in endothelial cells. J Biol Chem. 2010; 285 (51): 40104-40113.
48. Kelland L R. Preclinical perspectives on platinum resistance. Drugs. 2000; 59 Suppl 4:1-38.
49. Zsengellér Z K, Ellezian L, Brown D, et al. Cisplatin nephrotoxicity involves mitochondrial injury with impaired tubular mitochondrial enzyme activity. J Histochem Cytochem. 2012; 60 (7): 521-529.
50. Galgamuwa R, et al. Dichloroacetate prevents cisplatin-induced nephrotoxicity without compromising cisplatin anticancer properties. J Am Soc Nephrol. 2016; 27 (11): 3331-3344.
51. Cetin R, Devrim E, Kiliçoğlu B, Avci A, Candir O, Durak I. Cisplatin impairs antioxidant system and causes oxidation in rat kidney tissues: possible protective roles of natural antioxidant foods. J Appl Toxicol. 2006; 26 (1): 42-46.
52. Estrela G R, et al. MATE-1 modulation by kinin B1 receptor enhances cisplatin efflux from renal cells. Mol Cell Biochem. 2017; 428 (1-2): 101-108.
53. Nakamura T, et al. Disruption of multidrug and toxin extrusion MATE1 potentiates cisplatin-induced nephrotoxicity. Biochem Pharmacol. 2010; 80 (11): 1762-1767.
54. Kim D G, Bynoe M S. A2A adenosine receptor modulates drug efflux transporter P-glycoprotein at the blood-brain barrier. J Clin Invest. 2016; 126 (5): 1717-1733.
55. Menzies R I, Tam F W, Unwin R J, Bailey M A. Purinergic signaling in kidney disease. Kidney Int. 2017; 91 (2): 315-323.
56. Ohta A, et al. A2A adenosine receptor protects tumors from antitumor T cells. Proc Natl Acad Sci USA. 2006; 103 (35): 13132-13137.
57. Sitkovsky M V, et al. Hostile, hypoxia-A2-adenosinergic tumor biology as the next barrier to overcome for tumor immunologists. Cancer Immunol Res. 2014; 2 (7): 598-605.
58. Allard B, Allard D, Buisseret L, Stagg J. The adenosine pathway in immuno-oncology. Nat Rev Clin Oncol. 2020; 17 (10): 611-629.
59. Yu F, Zhu C, Xie Q, Wang Y. Adenosine A2A receptor antagonists for cancer immunotherapy. J Med Chem. 2020; 63 (21): 12196-12212.
60. Kawahara M, et al. Caffeine dose-dependently potentiates the antitumor effect of cisplatin on osteosarcomas. Anticancer Res. 2008; 28 (3A): 1681-1685.
61. Abe K, Yamamoto N, Hayashi K, Takeuchi A, Tsuchiya H. Caffeine citrate enhanced cisplatin antitumor effects in osteosarcoma and fibrosarcoma in vitro and in vivo. BMC Cancer. 2019; 19 (1): 689.
62. Chen J F, Cunha R A. The belated U S FDA approval of the adenosine A2A receptor antagonist istradefylline for treatment of Parkinson's disease. Purinergic Signal. 2020; 16 (2): 167-174.
63. Sharp C N, et al. Repeated administration of low-dose cisplatin in mice induces fibrosis. Am J Physiol Renal Physiol. 2016; 310 (6): F560-F568.
64. Sharp C N, Siskind L J. Developing better mouse models to study cisplatin-induced kidney injury. Am J Physiol Renal Physiol. 2017; 313 (4): F835-F841.
65. Chaplan S R, et al. Quantitative assessment of tactile allodynia in the rat paw. J Neurosci Methods. 1994; 53 (1): 55-63.
66. Laumet G, et al. Cisplatin educates CD8+ T cells to prevent and resolve chemotherapy-induced peripheral neuropathy in mice. Pain. 2019; 160 (6): 1459-1468.
67. Pellegrini K L, et al. MicroRNA-155 deficient mice experience heightened kidney toxicity when dosed with cisplatin. Toxicol Sci. 2014; 141 (2): 484-492.
68. Aschauer L, et al. Expression of xenobiotic transporters in the human renal proximal tubule cell line RPTEC/TERT1. Toxicol In Vitro. 2015; 30 (1 Pt A): 95-105.
69. Vandenbussche C, et al. Tacrolimus-induced nephrotoxicity in mice is associated with microRNA deregulation. Arch Toxicol. 2018; 92 (4): 1539-1550.
70. Lemaire J, et al. Cadmium-induced renal cell toxicity is associated with microrna deregulation. Int J Toxicol. 2020; 39 (2): 103-114.
71. Vomelová I, et al. Methods of RNA purification. All ways (should) lead to Rome. Folia Biol (Praha). 2009; 55 (6): 243-251.
72. Dobin A, et al. STAR: ultrafast universal RNA-seq aligner. Bioinformatics. 2013; 29 (1): 15-21.
73. Anders S, et al. HTSeq—a Python framework to work with high-throughput sequencing data. Bioinformatics. 2015; 31 (2): 166-169.
74. Anders S, Huber W. Differential expression analysis for sequence count data. Genome Biol. 2010; 11 (10): R106.
75. Love M I, Huber W, Anders S. Moderated estimation of fold change and dispersion for RNA-seq data with DESeq2. Genome Biol. 2014; 15 (12): 550.
76. Benjamini Y, Hochberg Y. Controlling the False Discovery Rate-a Practical and Powerful Approach to Multiple Testing. J R Stat Soc Ser B-Methodological. 1995; 57 (1): 289-300.
77. Heussner M J, et al. A novel syngeneic immunocompetent mouse model of head and neck cancer pain independent of interleukin-1 signaling. Anesth Analg. 2021; 132 (4): 1156-1163.

1. A method for the treatment of chemotherapeutic drug-induced nephrotoxicity in a subject in need therefore comprising administering to the subject a therapeutically effective amount of a selective A_{2A} Adenosine Receptor (A_{2A}R) antagonist.

2. The method according to claim 1 wherein the selective A_{2A} Adenosine Receptor antagonist is selected from the group consisting of istradefylline (KW-6002) or its derivatives, MSX-3, SCH-58261, tozadenant (SYN 115), NIR 178, ciforadenant, AB928, AZD4635, EOS100850, preladenant (SCH-420814), Inupadenant (EOS-850), EXS21546, TT-10 and TT-53 and pharmaceutically acceptable salts thereof.

3. The method according to claim 1 wherein the selective A_{2A} Adenosine Receptor antagonist is KW-6002 or a derivative thereof.

4. The method according to claim 1 wherein the selective A_{2A} Adenosine Receptor antagonist is administrated by oral or by parenteral administration.

5. A method for the treatment of cancer in a subject in need thereof comprising administering to the subject a therapeutically effective amount of a combination of a selective A_{2A} Adenosine Receptor antagonist and a chemotherapeutic drug.

6. The method according to claim 5 wherein the chemotherapeutic drug is a DNA-alkylating or a pharmaceutically acceptable salts thereof.

7. The method according to claim 5 wherein the chemotherapeutic drug is cisplatin.

8. The method according to claim 5 wherein the cancer is is of a histological type selected from the group consisting of: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; non encapsulating sclerosing carcinoma; adrenal cortical carcinoma; endometroid carcinoma; skin appendage carcinoma; apocrine adenocarcinoma; sebaceous adenocarcinoma; ceruminous; adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; Sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malign melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brennertumor, malignant; phyllodestumor, malignant; synovial sarcoma; mesothelioma, malignant; dysgerminoma; embryonal carcinoma; teratoma, malignant; strumaovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblasticodontosarcoma; ameloblastoma, malignant; ameloblasticfibrosarcoma; pinealoma, malignant; chordoma; glioma, malignant; ependymoma; ependymoma; protoplasmic astrocytoma; fibrillary astrocytoma; astroblastoma; medulloblastoma, glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocyticleukemia; mast cell leukemia; megakaryoblasticleukemia; myeloid sarcoma; and hairy cell leukemia.

9. A pharmaceutical composition comprising a therapeutically effective amount of a selective A_{2A} Adenosine Receptor (A2AR) antagonist and a chemotherapeutic drug.

10. The pharmaceutical composition according to claim 9 wherein the selective A_{2A} Adenosine Receptor (A2AR) antagonist is KW-6002 or a derivative thereof.

11. The pharmaceutical composition according to claim 9 wherein the chemotherapeutic drug is cisplatin.

12. (canceled)

13. (canceled)

14. The method according to claim 3, wherein the derivative is MSX-3 or SCH-58261.

15. The method according to claim 5 wherein the DNA-alkylating agent is a platin-containing chemotherapeutic drug.

16. The method according to claim 5 wherein the platin-containing chemotherapeutic drug is cisplatin, carboplatin, oxaliplatin, or a pharmaceutically acceptable salt thereof.

17. The method of claim 8, wherein the cancer is bladder, blood, bone, bone marrow, brain, breast, colon, oesophagial, gastrointestinal, gum, head, kidney, liver, lung, nasopharyngial, neck, ovary, prostate, skin, stomach, testis, tongue, or uterine cancer.
